(19) United States
(12) Patent Application Publication
(10) Pub. No.: US 2007/0135999 A1
(43) Pub. Date: Jun. 14, 2007

(54) Method, apparatus and system for characterizing pathological specimen

(75) Inventor: Tsafrir Kolatt, Haifa (IL)

Correspondence Address: Martin D. Moynihan;PRTSI, Inc., P.O. Box 16446, Arlington, VA, 22215, US

(73) Assignee: Applied Spectral Imaging Ltd., Migdal HaEmek (IL)

(21) Appl. No.: 11/480,480

(22) Filed: Jul. 5, 2006

Related U.S. Application Data
(60) Provisional application No. 60/749,589, filed on Dec. 13, 2005.

(51) Int. Cl. C12Q 1/00; G06F 19/00

(52) U.S. Cl. 702/19; 435/4

(57) ABSTRACT

A method for characterizing a stained pathological specimen is disclosed. The method comprises obtaining an image of the specimen, classifying the picture-elements of the image into classification groups, and using the classification groups to define at least one set of picture-elements corresponding to at least one tissue region of the pathological specimen. The method further comprises applying, on each set of picture-elements, at least one set-operator so as to characterize the tissue regions according to image data and spatial characteristics of the set.

### RELATED APPLICATION

This application claims the benefit of U.S. Provisional Patent Application No. 60/749,589, filed Dec. 13, 2005, the contents of which are hereby incorporated by reference.

### FIELD AND BACKGROUND OF THE INVENTION

The present invention relates to pathology and, more particularly, but not exclusively, to a method, apparatus and system for automatic characterization of stained pathological specimen.

The field of pathology involves the examination of tissue specimens to determine if the tissue is normal or diseased. The tissue specimens can be individual cells in a smear, body fluid or cell block (cytology specimens) or cell aggregates that form a structure with a specific function (histology specimens). Typically, pathology determines the structural and functional changes in cells, tissues and organs which cause or are caused by disease.

Histology serves as an invaluable tool in pathology since it deals with microanatomy of tissues and their cellular structure. Expressions of pathology are typically detected by the examination of histological sections of suspected tissues. A specimen is processed and applied to a microscope slide and then stained to make the normally transparent cells brilliantly colored for easier observation and to distinguish the various cellular elements which have differing affinities for the various stains such as Hematoxylin and Eosin, Fuchsin, Giemza, and the like. Different colors are thus associated with different tissue components. However, it is recognized that these stains are not always accurate because their appearance depends on many factors including solution preparation, environmental factors (temperature, etc.), co-existence of other stains, affinity of the stained element and the like.

In immunohistochemistry, spectrally marked antibodies are applied to the specimen to detect specific protein manifestations within the tissue thereby to obtain higher level of resolution compared to histology staining and information regarding their functionality. Over the past two decades, immunohistochemistry, which is also known as immunocytochemistry when applied to cells, has become an indispensable tool in diagnostic pathology and has virtually revolutionized the practice of surgical pathology. The terms immunohistochemistry and immunocytochemistry are used herein interchangeably.

Panels of monoclonal antibodies can be used in the differential diagnosis of undifferentiated neoplasms (e.g., to distinguish lymphomas, carcinomas, and sarcomas); to reveal markers specific for certain tumor types; to diagnose and phenotype malignant lymphomas; and to demonstrate the presence of viral antigens, oncoproteins, hormone receptors, and proliferation-associated nuclear proteins. Not only do such markers have diagnostic significance, but there is a growing body of evidence that some tumor markers have prognostic significance, as has been most extensively demonstrated, for example, in breast cancer. These marker studies can be performed by immunohistochemistry on a variety of specimen types, including cytological preparations, paraffin-embedded tissue and frozen sections. Most clinical assays employ a single antibody for each slide but by using chromogens of different colors, it is possible to observe/demonstrate two or more markers simultaneously. The specific (or primary) antibody may be labeled directly, or a second antibody carrying the label can be used to specifically bind to the first antibody.

Traditionally, the staining procedures are followed by a visual tissue inspection performed by the pathologist or, in case of genetic marking, by the cytogeneticist. The stained samples are typically compared to libraries, atlases or previous assays. Known in the art are atlases containing large number of samples both for “normal” or “abnormal” tissues. Tissue abnormalities can range from the mere expression of a certain protein via genetic to shape deformation of the tissue or tissue elements. Proliferation or deficiencies of these components possibly point at benign or pathologic condition of the tissue. Based on the visual inspection and comparison to other cases, the pathologist or cytogeneticist provides diagnosis, prognosis and for some cases even the adequate therapy.

It is recognized, however, that visual tissue inspection suffer from many limitations. For example, in pathology diagnostic of skin lesions the samples include many similar tissue elements and even a highly experienced pathologist finds it difficult to differentiate between one tissue element to the other. Studies show that skin lesion diagnoses performed by different pathologists for different sections of the same lesion may diverse to a great extent [Ackerman A. B, 1996, “Discordance among expert pathologists in diagnosis of melanocytic neoplasmas,” Hum. Pathol. 30:513-520; Corona et al., 1996, “Interobserver variability on the histopathologic diagnosis of cutaneous melanoma and other pigmented skin lesions,” J Clin Oncol. 14(4):1218-1223; Farmer et al., 1996, “Discordance in the histopathologic diagnosis of melanoma and melanocytic nevi between expert pathologists,” Hum Pathol. 27(6): 528-531; Cook et al., 1997, “The evaluation of diagnostic and prognostic criteria and the terminology of thin cutaneous malignant melanoma by the CRC Melanoma Pathology Panel,” Histopathology 30(2):195-197; CRC Melanoma Pathology panel, 1997, “A nationwide survey of observer variation in the diagnosis of thin cutaneous malignant melanoma including the MIN terminology,” Journal of Clinical Pathology 50:202-205]. The difficulty in providing a reliable diagnostic was also demonstrated in a study perfumed by Farmer [Farmer R. E., 2002, “The fundamental issue of diagnosis,” Archives of Dermatology 138(5): 684] which showed significant deviations between diagnoses given by the same pathologist to different sections of the same pathological sample.

Pathology reports regarding tissue condition are mainly descriptive. Even when the report includes quantitative measures relevant to the specific diagnosis, these measures are estimated by the examining pathologist and are rarely measured by objective means. Although this method bears its own merits, the disadvantages of such reports are evident. They are subjective to a large extent, irreproducible, do not always use the same reference line and cannot be compared to each other.

Although the pathologists' experience often helps them to determine morphological deformations that occur within the tissue, such determination is far from being optimal. The human eye has limited abilities in whatever concerns quantification of aggregation, correlation, alignment and the like, especially when irrelevant data and noise are present. Additionally, it is recognized that prior knowledge oftentimes leads to human biases.

In the past few years, attempts have been made to perform part of the analysis automatically. For example, known in the art are automatic methods for identifying and counting the fluorescent marks. However, the processed results still need to be manually examined by the cytogeneticists for their review and confirmation. Moreover, complicated tasks, such as the quantification of the immunohistochemical slides, remain manual tasks performed in slow and tedious processes.

One of the simplest tasks in cytological or histological diagnosis is counting. Counting can provide binary information (e.g., whether or not a protein is expressed or a gene is detected), or more quantitative information in which the level of protein expression is provided, in the form of, for example, number density (occurrences per unit area or volume), number density within specific tissue regions (e.g., malignant nests), and the like. The quantitative information can also include additional expression level information such as dye affinity reflecting protein expression in a single cell or a set of cells.

Known in the art is a counting method called fluorescent in-situ hybridization (FISH), in which a ratio count of fluorescent dots, with respect to control set of differently colored dots, is used to quantify the tissue condition. Also known are variants of the FISH method, including M-ISH, ISH and the like. However, although the counting is a relatively simple task, the prior art techniques suffer from many complications such as insufficient sensitivity, saturation, accidental dots etc. In particular the prior art is incapable of handling complicated situations, including diverge signals, spatial arrangement effects, application of various conditions (e.g., shape, size ratios) and the like.

Although image analysis can aid for circumventing certain steps in the identification process (to this end see, e.g., U.S. Pat. Nos. 4,017,192, 6,718,053, 6,697,509, 6,631,203, and 6,920,239), manual supervision, review and oftentimes intervention is inevitable.

To date, most characterizations of the tissue specimen or specific elements thereof (e.g., cells with protein expression) are performed by visual inspection and the pathology results are described in a qualitative manner. In cases where there is a standard gamut for, e.g., the protein expression level, the expert estimates the level with respect to this scale. In cases in which the tissue architecture provides information regarding a certain disorder, the expert depicts the findings with respect to the protocol items of the specific syndrome or disorder. Whenever the analysis includes inter-relationships between different elements of the tissue, the analysis is described qualitatively, rather than quantitatively.

Computer aided diagnosis (CAD) is a known field for automated analysis of imagery information (to this end see, e.g., Huang, K. and F. R. Murphy, 2004, “From quantitative microscopy to automated image understanding,” Journal of Biomedical Optics 9(5):893-912). It is recognized that CAD pathology has the potential of providing at least partial quantitative analyses. Several attempts have been made to adopt CAD for pathology, typically in the area known as morphometry (see, e.g., U.S. Pat. No. 5,991,028; Kosma et al., 1985, “Reproducibility and variation of morphometric analysis of carcinoembryonic antigen staining in histopathology. The influence of standardized microscopic fields,” Analytical & Quantitative Cytology & Histology 7: 271-274; Laitakari, J., 2003, “Computer-Assisted quantitative Image analysis of Cell Proliferation, Angiogenesis and Stromal Markers in Experimental and Laryngeal Tumor Development,” Acta Univ. Oul. D 713; and Erdkamp et al., 1994, “Comparison of image (CAS 200) and flow cytometry determined DNA content of paraffin-embedded Hodgkin's disease tissue,” Hematologic Pathology 8: 75-84). The use of CAD for pathology has shown that a simple measure (such as size, shape, ratios) is strongly correlated with tumor types [Gil et al., 2002, “Image analysis and morphometry in the diagnosis of breast cancer,” Microscopy Research and Technique 59: 109-118]

Prior art CAD pathology, however, had very limited success. For example, the prior art failed to segregate between different tissue elements and identify small differences in components characterizations.

There is thus a widely recognized need for, and it would be highly advantageous to have a method, apparatus and system for characterizing a pathological specimen, devoid of the above limitations.

### SUMMARY OF THE INVENTION

According to one aspect of the present invention there is provided a method of analyzing an image of a stained pathological specimen. The image is arranged gridwise in a plurality of picture-elements, each being associated with image data over a grid. The method comprises: defining at least one set of picture-elements over the grid, and applying, on each set of picture-elements, at least one set-operator, wherein each set-operator is associated with a predetermined diagnosis describing the pathological specimen, thereby analyzing the image.

According to further features in preferred embodiments of the invention described below, the method further comprises issuing a report describing the pathological specimen, based on results obtained by the application of the at least one set-operator.

According to another aspect of the present invention there is provided a method of characterizing a stained pathological specimen. The method comprises: obtaining an image of the specimen; classifying the picture-elements into classification groups according to the image data; using the classification groups to define at least one set of picture-elements corresponding to at least one tissue region of the pathological specimen; and applying, on each set of picture-elements, at least one set-operator so as to characterize the tissue regions according to image data and spatial characteristics of the set.

According to further features in preferred embodiments of the invention described below, the method further comprises using the classification groups to define at least one set of picture-elements corresponding to at least one background region of the pathological specimen.

According to still further features in the described preferred embodiments the definition of the set(s) of picture-elements comprises clustering at least a portion of the picture-elements according to the classification groups, thereby providing at least one cluster of picture-elements.

According to still further features in the described preferred embodiments the application of the set-operator(s) on each the set of picture-elements, comprises applying the set-operator(s) on the cluster(s) of picture-elements.

According to still further features in the described preferred embodiments the definition of the set(s) of picture-elements comprises applying a geometrical modeling procedure to at least a portion of the plurality of picture-elements.

According to still further features in the described preferred embodiments the definition of the set(s) of picture-elements comprises applying a geometrical modeling procedure to the cluster(s) of picture-elements.

According to still further features in the described preferred embodiments the method further comprises normalizing the image data prior to the classification.

According to still further features in the described preferred embodiments the method further comprises combining at least a portion of the classification groups.

According to still further features in the described preferred embodiments the method further comprises employing at least one counting technique to the stained pathological specimen, and correlating the results of the counting technique with the set(s) of picture-elements.

According to yet another aspect of the present invention there is provided apparatus for characterizing a stained pathological specimen based on an image of the specimen. The apparatus comprises: classification unit, for classifying the picture-elements into classification groups according to the image data; a set definition unit, for defining at least one set of picture-elements corresponding to at least one tissue region of the pathological specimen, using the classification groups; a data analysis unit, for applying at least one set-operator on each set of picture-elements, so as to characterize the tissue regions according to image data and spatial characteristics of the set, thereby characterizing the pathological specimen.

According to still another aspect of the present invention there is provided a system for characterizing a stained pathological specimen, comprises an imaging apparatus, for providing the image of the specimen, and the apparatus described above.

According to further features in preferred embodiments of the invention described below, the set definition unit is operable to define at least one set of picture-elements corresponding to at least one background region of the pathological specimen, using the classification groups.

According to still further features in the described preferred embodiments the apparatus and/or system further comprises a clustering unit, for clustering at least a portion of the picture-elements according to the classification groups, to provide at least one cluster of picture-elements.

According to still further features in the described preferred embodiments the data analysis unit is operable to apply the set-operator(s) on the cluster(s) of picture-elements.

According to still further features in the described preferred embodiments the apparatus and/or system further comprises a geometrical modeling unit for applying a geometrical modeling procedure to at least a portion of the plurality of picture-elements.

According to still further features in the described preferred embodiments the apparatus further comprises further comprises a geometrical modeling unit for applying a geometrical modeling procedure to the cluster(s) of picture-elements.

According to still further features in the described preferred embodiments the classification unit is operable to normalize the image data. According to still further features in the described preferred embodiments the classification unit is operable to combine at least a portion of the classification groups.

According to still further features in the described preferred embodiments the cluster(s) comprises at least one sub-cluster.

According to still further features in the described preferred embodiments the geometrical modeling is applied to the at least one sub-cluster.

According to still further features in the described preferred embodiments the cluster(s) comprises at least one cluster of background picture-elements.

According to still further features in the described preferred embodiments the set(s) of picture-elements is defined by cross correlation of at least one cluster of background picture-elements with at least one cluster of non-background picture-elements.

According to still further features in the described preferred embodiments the image comprises a spectral image and the image data comprises a wavelength spectrum.

According to still further features in the described preferred embodiments the image comprises a monochrome image and the image data comprises intensity values.

According to still further features in the described preferred embodiments the classification groups are selected from a predefined set of classification groups, each being associated with a predetermined wavelength spectrum.

According to still further features in the described preferred embodiments the classification groups are defined based on the wavelength spectra of the picture-elements.

According to still further features in the described preferred embodiments the classification groups are defined iteratively.

According to still further features in the described preferred embodiments the classification groups are defined non-iteratively.

According to still further features in the described preferred embodiments the set-operator(s) comprises an operator for calculating statistical distributions.

According to still further features in the described preferred embodiments the set-operator(s) comprises an operator for calculating statistical moments.

According to still further features in the described preferred embodiments the set-operator(s) comprises an operator for calculating tensor of inertia.

According to still further features in the described preferred embodiments the set-operator(s) comprises an operator for calculating distribution of parameters obtained from the geometrical modeling procedure.

According to still further features in the described preferred embodiments the set-operator(s) comprises an operator for calculating coordinates.

According to still further features in the described preferred embodiments the set-operator(s) comprises an operator for calculating an average normalization factor over the cluster(s) of picture-element.

According to still further features in the described preferred embodiments the set-operator(s) comprises an operator for calculating population characteristics of the set(s) of picture-elements, hence to provide a population map characterizing the stained pathological specimen.

According to still further features in the described preferred embodiments the method further comprises employing at least one counting technique to the stained pathological specimen, to provide an amplification map characterizing the stained pathological specimen, and correlating the amplification map with the population map.

According to still further features in the described preferred embodiments the spectral image is characterized by two spatial dimensions.

According to still further features in the described preferred embodiments the spectral image is characterized by three spatial dimensions.

According to still further features in the described preferred embodiments the image comprises a set of spectral images and the image data comprises a wavelength spectrum.

According to still further features in the described preferred embodiments at least two images of the set of spectral images are characterized by a different magnification level.

According to still further features in the described preferred embodiments at least two images of the set of spectral images are captured following a different staining of the pathological specimen.

According to still further features in the described preferred embodiments at least two images of the set of spectral images are captured by a different illumination scheme.

According to still further features in the described preferred embodiments at least two images of the set of spectral images are captured by a different spectral acquisition scheme.

According to still further features in the described preferred embodiments at least two images of the set of spectral images correspond to different region-of-interests of the pathological specimen.

According to still further features in the described preferred embodiments the pathological image is stained with a stain selected from the group consisting of a direct immunohistochemical stain, a secondary immunohistochemical stain, a histological stain, immunofluorescence stain, a DNA ploidy stain, a nucleic acid sequence specific probe and any combination thereof.

According to still further features in the described preferred embodiments the pathological image is stained using a method selected from the group consisting of Romanowsky-Giemsa staining, Haematoxylin-Eosin staining and May-Grunwald-Giemsa staining.

The present invention successfully addresses the shortcomings of the presently known configurations by providing method, apparatus and system suitable for analyzing an image and/or characterizing a stained pathological specimen.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

Implementation of the method and system of the present invention involves performing or completing selected tasks or steps manually, automatically, or a combination thereof. Moreover, according to actual instrumentation and equipment of preferred embodiments of the method and system of the present invention, several selected steps could be implemented by hardware or by software on any operating system of any firmware or a combination thereof. For example, as hardware, selected steps of the invention could be implemented as a chip or a circuit. As software, selected steps of the invention could be implemented as a plurality of software instructions being executed by a computer using any suitable operating system. In any case, selected steps of the method and system of the invention could be described as being performed by a data processor, such as a computing platform for executing a plurality of instructions.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a flowchart diagram of a method suitable for characterizing a stained pathological specimen, according to various exemplary embodiments of the present invention;

FIG. 2 is a schematic illustration of an apparatus for characterizing a stained pathological specimen, according to various exemplary embodiments of the present invention;

FIG. 3 is a schematic illustration of a system for characterizing a stained pathological specimen, according to various exemplary embodiments of the present invention;

FIGS. 4a-b show a skin lesion section superimposed by principal axes, calculated according to a preferred embodiment of the present invention;

FIG. 5a shows cell nuclei field (red) superimposed over a color image of a skin lesion section, stained by Hematoxylin and Eosin;

FIG. 5b is a histogram showing the probability distribution of the nucleus eccentricity of the image of FIG. 5a, as calculated according to according to a preferred embodiment of the present invention;

FIG. 6a shows probability distribution for clusters defining nuclei as a function of the logarithm of the size of the nuclei, calculated for 5 different skin lesions under ×20 magnification according to a preferred embodiment of the present invention;

FIG. 6b shown the probability of finding N nuclei within a square with a linear dimension of 20 microns, calculated for a skin lesion under ×20 magnification according to a preferred embodiment of the present invention;

FIG. 6c shows a weighted (blue) and un-weighted (red) distance correlation function of cell nuclei for a skin lesion section under ×20 magnification, according to a preferred embodiment of the present invention;

FIGS. 7a-d show color images of two skin lesion sections under ×20 magnification (FIGS. 7a-b), and two distance correlation functions calculated according to a preferred embodiment of the present invention for each skin lesion section (FIGS. 7c-d);

FIGS. 8a-b show a skin lesion section under ×20 magnification (FIG. 8a) and the average linear size of nuclei in the skin lesion section calculated according to a preferred embodiment of the present invention as function of their distance ρ from the upper edge of the section (FIG. 8b);

FIG. 9 shows a tissue biopsy taken from a woman's breast and stained with ki-67 staining;

FIG. 10 shows a rat heart tissue section stained for epithelial cell identification (brown) with Hematoxylin counter stain;

FIG. 11 shows a tumor tissue which was subjected to a blood vessel density analysis, according to a preferred embodiment of the present invention;

FIG. 12 shows a typical skewness distribution for skin lesion sections under low (×2) magnification, calculated according to a preferred embodiment of the present invention; and

FIGS. 13a-b show examples of the bi-variant distribution of skin lesion nuclei eccentricity and goodness-of-fit of a geometrical model, calculated according to a preferred embodiment of the present invention.

### DESCRIPTION OF THE PREFERRED EMBODIMENTS

The present invention is of a method, apparatus and system which can be used in pathology. Specifically, the present invention can be used to automatically characterize stained pathological specimens by image analysis.

The principles and operation of a method, apparatus and system according to the present invention may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

The method, apparatus and system of the present embodiments are suitable for characterizing a stained pathological specimen. The pathological specimen can be of any type, including, without limitation, a histological slide, an immunohistochemical slide, an in-situ hybridization (ISH) slide (e.g., a FISH slide, an M-ISH slide, etc.) and any combination thereof.

As used herein in the specification and in the claims section below, the term “stained” or “staining” refers to a process in which coloration is produced by foreign matter having penetrated into and/or interacted with the pathological specimen.

The specimen can be stained in any way known in the art, including, without limitation, via immunohistochemical stain, a histological stain, a DNA ploidy stain, nucleic acid (DNA or RNA) sequence specific probes (from single locus, gene or EST sequence to whole chromosome or chromosomes paints) or any combination thereof. The histological stain can be, for example, Hematoxylin-Eosin stain, Giemsa stains of different types (Romanowsky-Giemsa, May-Grunwald-Giemsa, etc.), Masson's trichrome, Papanicolaou stain and the like.

As used herein in the specification and in the claims section below, the term “stain” or “stains” refers to colorants, either fluorescent, luminescent and/or non-fluorescent (chromogenes) and further to reagents or matter used for effecting coloration.

As used herein in the specification and in the claims section below, the term “immunohistochemical stain” refers to colorants, reactions and associated reagents in which a primary antibody which binds a cytological marker is used to directly or indirectly (via “sandwich” reagents and/or an enzymatic reaction) stain the biological sample examined. Immunohistochemical stains are in many cases referred to in the scientific literature as immunostains, immunocytostains, immunohistopathological stains, etc.

As used herein in the specification and in the claims section below, the term “histological stain” refers to any colorant, reaction and/or associated reagents used to stain cells and tissues in association with cell components such as types of proteins (acidic, basic), DNA, RNA, lipids, cytoplasm components, nuclear components, membrane components, etc. Histological stains are in many cases referred to as counterstains, cytological stains, histopathological stains, etc.

As used herein in the specification and in the claims section below, the term “DNA ploidy stain” refers to stains which stoichiometrically bind to chromosome components, such as, but not limited to, DNA or histones. When an antibody is involved, such as anti-histone antibody, such stains are also known as DNA immunoploidy stains.

Lists of known stains are provided in U.S. Pat. No. 6,007,996, filed Jul. 27, 1998, the contents of which are hereby incorporated by reference.

As used herein in the specification and in the claims section below, the phrase “nucleic acid sequence specific probe” refers to polynucleotides labeled with a label moiety which is either directly or indirectly detectable, which polynucleotides being capable of base-pairing with matching nucleic acid sequences present in the biological sample.

Before providing a detailed description of the method, apparatus and system for characterizing stained pathological specimen in accordance with the present embodiments, attention will be given to the advantages and potential applications offered thereby.

The present embodiments successfully characterize the stained pathological specimen in a quantitative and preferably automatic manner with minimal or no visual inspection. The present embodiments therefore useful for performing automatic quantitative pathology diagnostics. The present embodiments make use of the observation that tissues are made of compound structures and multiple components (tissue elements), and employ a novel characterization approach which differs from an element-by-element description. The present embodiments are particularly useful for the characterization of ensembles of components such as, but not limited to, cells, extra-cellular matrix, blood vessels, nuclei, and the like, for which the statistical approach of the present embodiments provides high quality quantitative results.

The characterization of the pathological specimen according to the present embodiment is based on the definition of sets on the one hand, and the use of mathematical operations on the sets on the other hand. This allows the present embodiments to characterize stained pathological specimen and to overcome various problems such as measurement noise, finite numbers, sparse and finite sampling, varying magnification and/or resolution rates, inhomogeneities in the bright-field or excitation illumination. The present embodiments also overcome the problems of stain fade-out and variability within a specific slide, across slides of the same tissue sample, between different tissues, and across time.

In various exemplary embodiments of the invention the method, apparatus and system are capable of recognizing shapes and assessing the quality of the shape recognition by attaching errors to each measurement. The mathematical operations performed on sets according to the present embodiments can uncover hidden structure within the tissue. Particularly, but not exclusively, the present embodiments are advantageous in cases in which it is difficult to perform visual diagnosis and the pathologist is in search for supportive evidence to determine the tissue status. For example, in early stage cancer or in malignant tumors resembling benign tumors or vice versa, the present embodiments can be used to accurately characterize the pathological specimen by revealing structures which cannot be inspected visually.

In various exemplary embodiments of the invention the method, apparatus and system are capable of distinguishing between plasma and nuclei in cells, identifying foreign bodies, pointing out irrelevant tears which may be present due to the preparation procedure and to distinguish between different tissue regions.

An additional advantage of the present embodiments is the ability to distinguish between different elements marked by the same stain. Although stains are designed to bind to specific tissue elements, they do not always bind exclusively to the target element and residual stains can be found on other elements and on top of the counter stain. In various exemplary embodiments of the invention the method, apparatus and system identify residual stains in the specimen and distinguish them from the target elements.

The method apparatus and system of the present embodiments can thus be used by the pathologist to characterize the pathological specimen and provide accurate description and diagnosis. The pathologist can stain the specimen with the desired stains and perform the characterization procedure using the method, apparatus and/or system of the present embodiments, taking into account the desired type diagnosis (e.g., density of blood vessel, identification of suspected nevus section, etc.).

The method, apparatus and system of the present embodiments preferably provides the pathologist with a report which can be, for example, in the form of characteristic numbers or functions. The numbers and functions can be attributed to the specific diagnosis queries (e.g., malignancy of a specific tumor) by showing their location within predetermined parameters distributions. In the simplest case, the characteristic numbers or functions are projected onto a binary distribution of “true” or “false” for each specific diagnosis query. In other embodiments, the characteristic numbers or functions provide statistical or probabilistic information (e.g., malignancy level) for the diagnosis query.

The results provided by the method, apparatus or system of the present embodiments can be stored in the database. Additional information obtained by other means (e.g., genetic test or the like) can be added to the same database. The accumulated data in the database can then be further processed to update the attribution of the characteristic numbers or functions to the diagnosis queries.

The method, apparatus and system of the present embodiments can serves as a CAD tool for existing pathological diagnostics as well as a research tool for finding hidden structure and correlations between tissue characteristics and clinical status. Moreover, data accumulation on similar tissue analyses performed using the method, apparatus and system of the present embodiments can be used for constructing a database which in turn can be used for comparing different tissue appearances on a common reference frame.

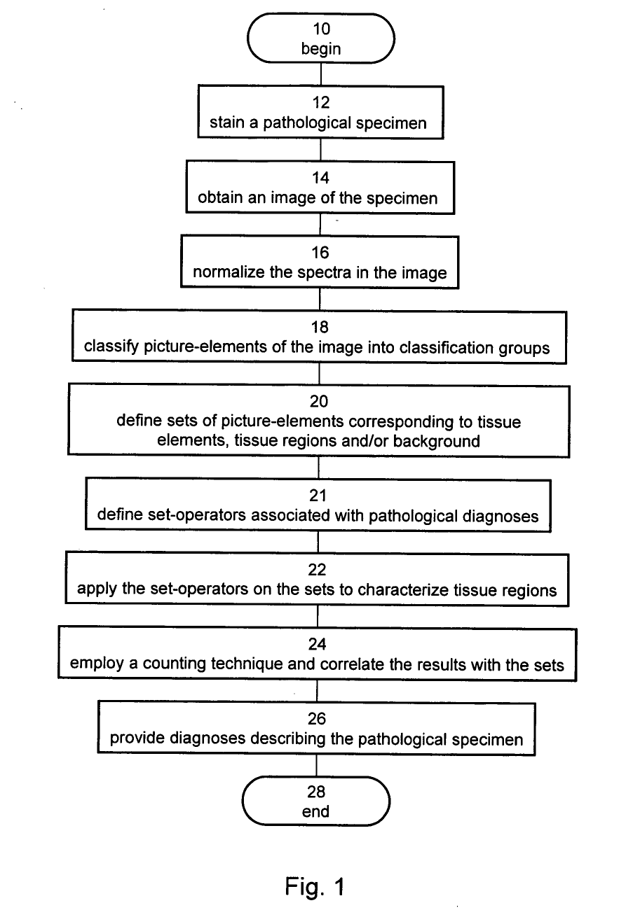

Referring now to the drawings, FIG. 1 is a flowchart diagram of the method according to various exemplary embodiments of the present invention.

It is to be understood that, unless otherwise defined, the method steps described hereinbelow can be executed either contemporaneously or sequentially in many combinations or orders of execution. Specifically, the ordering of the flowchart of FIG. 1 is not to be considered as limiting. For example, two or more method steps, appearing in the following description or in the flowchart of FIG. 1 in a particular order, can be executed in a different order (e.g., a reverse order) or substantially contemporaneously. Additionally, one or more method steps appearing in the following description or in the flowchart of FIG. 1 are optional and are presented in the cause of providing a useful description of an embodiment of the invention. In this regard, there is no intention to limit the scope of the present invention to any of the method steps presented in FIG. 1.

The method begins at step 10 and, optionally and preferably, continues to step 12 in which the pathological specimen is stained as further detailed hereinabove. The method continues to step 14 in which a spectral image or a monochrome image of the specimen is obtained. The image is arranged gridwise in a plurality of picture-elements (e.g., pixels, group of pixels, voxels, group of voxels and the like), respectively representing a plurality of spatial locations of the specimen. The spatial locations can be arranged over the specimen using a two-dimensional coordinate system to provide an image characterized by two spatial dimensions, or a three-dimensional coordinate system to provide an image characterized by three spatial dimensions. Each picture-element of the image is associated with image data depending on the type of image. For monochrome images, the image data in each picture-element comprises an intensity value or a grey-level. For spectral images, the image data of each picture-element comprises a wavelength spectrum which is typically in a form of a plurality of discrete intensity values, one intensity value for each wavelength in the spectrum.

The spectral or monochrome image can be a single image or, more preferably, a set of images wherein each image of the set corresponds to a different portion of the specimen, a different magnification, a different staining procedure, a different illumination scheme (transmission, reflectance, fluorescence, light source), a different spectral acquisition scheme (e.g., grey level, filters, hyper-spectral), a different z-layer and the like.

The terms “image” and “set of images” are interchangeably used in this document. In most cases the term “image” is used to indicate a set of images, however, this is not intended to limit the scope of the present invention, which embraces the use of any number of images.

The use of set of images has the advantage of significantly increasing the amount of information which can be extracted from the pathological specimen. This is particularly useful when the image are analyzed by employing statistical operators because, larger amount of information reduces the statistical errors hence improves the reliability of the specimen characterization.

Different magnification levels can be used for unveiling different types of hidden structures in the specimen. This is because different types of structures oftentimes involve different resolution levels. Thus, low magnification level can be used, e.g., for determining global symmetries within the specimen, and higher magnification levels can be used, e.g., for determining short-range correlations.

Images of portions of the specimen are optionally and preferably used when the magnification levels are larger than 100%. In these embodiments, different images of the sets preferably correspond to different regions-of-interest of the specimen. For example, a suitable region-of-interest for determining short range correlation between cells can encompass linear distance which is about one order of magnitude longer than the inter-cellular average distance. For nucleoli identification or FISH counting, for which the magnification level is typically from about 1 to about 2 orders of magnitude higher, the typical region-of-interest preferably encompasses linear distances which are of the order of the inter-cellular average distance or less.

As used herein the term “about” refers to ±10%. As used herein a quantity X is said to be of the order of Y, if the ratio X/Y is between about 0.1 and about 10.

A set of images of different magnification levels enables the foundation of large- and short-range quantities that can be matched in overlapping regions of the images. The magnification can also be chosen selectively by information extracted from either low-magnification or high-magnification images so as to capture complementary images in a non-random fashion. The use of a plurality of different magnification levels can also be used for calculating fractal dimensions, for example, to analyze and characterize the borderline between two adjacent tissue regions.

Each image in the set of images can be presented mathematically as A^{m,n}, where n is an image index and m is a magnification level index. Although not obligatory, the differentiation between the magnification level index and the image index is advantageous because, unlike the other types of images (different portions, illumination schemes, z-layers, etc.), geometric properties scale in proportion to the magnification an a-priori known manner. It is appreciated that different mathematical descriptions, in which each image in the set is represented using a different number of set-indices (e.g., a single set-index), is equivalent to this description. One of ordinary skills in the art, provided with the details described herein would know how to modify the present mathematical description according to the selected choice of representation.

The set of images is denoted A and generally defined as:

$$A = {Y}_{m , n}^{{N}_{img}} {A}^{m , n} , \qquad ( EQ . 1 )$$

where N_{img} is the number of images.

The specimen is composed of picture-element a^{m,n}, such that

$${A}^{m , n} = {Y}_{i , j} {a}_{i , j}^{m , n} , \qquad ( EQ . 2 )$$

where each picture-element is identified by the indices of the image to which it belongs, and by an identifier within the image, denoted (i,j). One skilled in the art will recognize that the picture-elements in Equation 2 are conveniently identified using a two-dimensional spatial coordinate system. It is nevertheless not intended to limit the scope of the present invention to two-dimensional spatial representation. All types of picture-elements identifiers within the image are contemplated, either spatial identifiers or non spatial identifiers (e.g., coordinates on a polarization plane), in any number of dimensions.

As stated, each picture-element is associated with image data. For monochrome image, a_{i,j}^{m,n} is associated with an intensity value u. For spectral images, a_{i,j}^{m,n} is associated with a discrete spectrum, [S_{i,j}^{m,n}(λₖ)], which is typically accompanied with the derived spectrum errors, e_{S}_{ᵢⱼ}. Without loss of generality, it is assumed that the spectrum does not depend on the magnification level. The magnification level index will therefore be omitted from the spectrum in part of the following description for clarity of presentation.

Without limiting the scope of the present invention, the following description will consider each picture-element as being associated with a series of N_{wl} intensity values corresponding to N_{wl} fixed wavelengths, λₖ (k=1, 2, . . . , N_{wl}). In practice, each such intensity value is measured through a filter having a known central wavelength λₖ. For simplicity, the light source spectrum is assumed to be flat. It will be appreciated that the spectrum of the light source can also have any other form, and one of ordinary skill in the art, provided with the details described herein would know how to adjust the process of the present embodiments in accordance with the form of the light source spectrum.

In various exemplary embodiments of the invention, the method continues to an optional step 16 in which the spectra of the picture-elements are normalized. Normalization of spectra is a well known procedure in which each spectrum, S_{i,j}^{m,n} is normalized using a normalization factor, R_{i,j}^{m,n}. A normalized spectrum is denoted herein by Ś_{i,j}^{m,n} and defined as:

$${{S}^{′}}_{i , j}^{m , n} = {S}_{i , j}^{m , n} / {R}_{i , j}^{m , n} \qquad ( EQ . 3 )$$

According to a preferred embodiment of the present invention R_{i,j}^{m,n} is calculated using the following equation:

$${R}_{i , j}^{m , n} = {\sum }_{{λ}_{k} = {λ}_{L}}^{{λ}_{H}} {S}_{i , j}^{m , n} ( {λ}_{k} ) , \qquad ( EQ . 4 )$$

where λ_{L} is a predetermined low wavelength limit and λ_{H} is a predetermined high wavelength limit. Other normalization definitions utilize, e.g., an integral of the interpolated spectrum function, \tilde{S}_{i,j}^{m,n}:

$${R}_{i , j}^{m , n} = {\int }_{{λ}_{L}}^{{λ}_{H}} {\tilde{S}}_{i , j}^{m , n} ( λ ) ⅆ λ . \qquad ( EQ . 5 )$$

In various exemplary embodiments of the invention the method continues to step 18 in which the picture-elements of the image (or set of images) are classified into classification groups. The classification groups are defined in accordance with the image data associated with each picture-element. Specifically, when the image is a monochrome image, the picture-elements are classified by their intensity value or grey-level. For example, for an 8-bit grey-scale image, there are 2⁸=256 different possible intensity values, and, theoretically, a total number of 256 classification groups can be defined (one classification group for each intensity value). In practice, however, the number of classification groups is smaller than the total number of possible intensity values, such that each classification group is associated with a range intensity values.

When the image is a spectral image, the picture-elements are classified by their spectra. Preferably, the classification step utilizes statistical analysis in which the resemblance between spectra is calculated to enable identification of multiple different spectra in the image.

The resemblance between two measured spectra is defined as the probability that the two measured spectra are drawn from the same underlying spectrum. Typically the resemblance is calculated using the statistical errors associated with the measured intensities at the respective wavelengths. The statistical errors are due to the finite number of photons, the wavelength bin size (and shape) and various environmental parameters, such as illumination, contamination, and the like. The calculation can be performed using any statistical test known in the art, including, without limitations, χ² test, goodness of fit, Kolmogorov-Smirnoff test and the like.

The detailed calibration of the resemblance parameter(s) is preferably performed in accordance with the tissue elements that need to be distinguished from one another, the staining quality and/or the staining variation.

The spectral classification can be executed in more than one way. For example, in one preferred embodiment, a set of classification groups is defined in advance, wherein each classification group is associated with a predetermined wavelength spectrum, and the method determines, for each picture-element, to which classification group it belongs. Specifically, each picture-element is analyzed according to a resemblance criterion in which the wavelength spectrum of the respective picture-element is compared to the wavelength spectra associated with the classification groups. The picture-element is then affiliated with the classification group for which the resemblance criterion is met. The resemblance criterion can be an absolute criterion (e.g., predetermined χ² per degrees of freedom threshold) or a relative criterion (e.g., minimal χ² per degrees of freedom over the image). In this embodiment, an additional group, referred to herein as the “orphans group,” is defined for all picture-elements that do not fall under the resemblance criterion to any of the classification groups in the predefined set.

In another preferred embodiment, the classification groups are not defined in advance, but rather defined dynamically during the classification step, based on the wavelength spectra of the existing picture-elements in the image. In this embodiment, the spectra are analyzed in view of the classification groups that have already been defined. Specifically, the spectrum associated with the first picture-element defines the first classification group, the spectrum associated with the second picture-element is compared with the spectrum associated with the first classification group and, if the resemblance criterion is met, the second picture-element is affiliated with the first classification group. If, on the other hand, the resemblance criterion is not met, a new classification group is defined. The comparison can be made either for each picture-element in which case the individual spectrum of the picture-element is used, or collectively for groups of adjacent picture-element, in which case the average spectrum of the group is used. In any event, the process is preferably repeated for all picture-elements wherein each picture-element is either affiliated (collectively or individually) with a previously defined classification group or defines (again, collectively or individually) a new classification group.

According to a preferred embodiment of the present invention for every addition of a picture-element or a group of adjacent picture-element to an existing classification group, the average spectrum and errors are re-calculated. Thus, during the entire process, each classification group is characterized by an average spectrum and error.

The classification groups can also be defined iteratively as follows. Once a set of classification groups is defined, it is considered as the zero's iteration, and all the picture-elements of the image are analyzed again in view of the zero's iteration. During the analysis, the method decides for each picture-element, to which of the classification groups of the zero's iteration it belongs. Every addition of a picture-element or a group of adjacent picture-elements to a classification groups is accompanied by a calculation of average spectrum and error, which calculation redefines the respective classification groups. Once all the picture-elements in the image are analyzed, a new set of classification groups is defined and considered as the first iteration. The iterative procedure is preferably repeated until the variations between successive iterations are sufficiently low (say, below 10%).

In an additional embodiment, the classification is performed by employing a regular or standard spectral un-mixing method, in which the spectrum of each picture-element is spectrum is represented in vector representation as a combination (e.g., linear combination in the logarithm) of basis vectors spanning the wavelength space. After representing all picture-elements using the basis vectors, the affiliation to the classification groups can be done by subjecting the coefficients of the vector representations to a threshold procedure. The basis vectors can be calculated using any method known in the art, such as, but not limited to, singular value decomposition and principal component analysis [to this end, see, e.g., U.S. Pat. No. 5,784,162 supra).

In various exemplary embodiments of the invention, at the end of the classification process (whether performed using a predefined set or a dynamically defined set of classification groups), similar classification groups are combined and picture-elements are re-distributed by associating each picture-element with a classification group using the same resemblance criterion and, optionally and preferably, with a different resemblance threshold.

The use of normalized spectra (see, e.g., Equations 3 and 4 above) during the spectral classification is advantageous because it reduces the effect of intensity variations due to temporal and spatial variation of the illumination, uneven staining, variability in optical depth (e.g., overlapping nuclei) and the like.

An additional advantage of the classification technique of the present embodiments is the reduction or elimination of the problem of uneven intensity in gray scale images. Since at least two wavelengths (“filters”) are considered, the normalized spectrum removes the degeneracy between uneven illumination (which results in an identical normalized spectrum) and true different objects with different spectra.

The method preferably continues to step 20 in which one or more sets of picture-elements are defined. Each set of picture-element can correspond to one or more tissue regions of the pathological specimen. Optionally and preferably, the method also defines one or more sets of picture-elements which correspond to one or more background regions.

The sets of picture-elements can be defined using any set of criteria for defining sets. For example, in the preferred embodiment in which the optional classification step (step 18) is executed, the sets of picture-elements can be defined based on their affiliation to the classification groups and their spatial locations. For example, the picture-elements or a portion thereof can be clustered according to the classification groups and a predetermined connectivity criterion. Each cluster is characterized by a spatial location via the connectivity of the picture-elements in the cluster and image data associated with the corresponding classification group. This is a generalization of the “segmentation-labeling” process. The preferred connectivity criterion is the picture-element proximity with or without diagonals.

The notation C_{n,k}^{m} represents the kth cluster of picture-element at magnification m and classification group n. In various exemplary embodiments of the invention the clustering is followed by a sub-clustering step in which additional criteria (typically, but not exclusively, geometrical criteria and/or intensity criteria) can be utilized. The sub-clustering step provides picture-element sets that in addition to their association with the parent cluster, fulfill the additional criteria. For example, sub-clustering of C_{n,k}^{m} can be performed by selecting all picture-element of C_{n,k}^{m} which also belong to a different cluster (or clusters) at different magnification. The sub-clustering can be iteratively repeated any number of times. Specifically, sub-clusters can also be subjected to sub-clustering procedures. The sub-clustering step thus provides a cluster hierarchy of any number of levels. The notation C_{n,k,i}^{m} represents is the ith sub-cluster of cluster k, hence C_{n,k,i}^{m}∈C_{n,k}^{m}.

Sets or clusters (including sub-clusters) can correspond to any regional component in the pathological specimen, including, without limitation, cells, nuclei, nucleoli, specific tissue (e.g., a blood vessel, a dermis, an epidermis), connecting tissue and the like.

In various exemplary embodiments of the invention a geometrical modeling procedure is applied to the picture-elements of the set or cluster or a portion thereof. The purpose of the geometrical modeling procedure is to identify geometrical shapes formed by picture-elements of the same classification group.

The advantage of using the geometrical modeling is that it allows the identification of different types of tissues or parts of tissues based on the knowledge of their shape. Additionally, the geometrical modeling provides a reference frame even when several tissue components appear to be different from one another. Moreover, the geometrical modeling allows an overall estimation of the error involved and therefore provides more reliable statistical estimates.

The geometrical modeling procedure can be applied to the picture-element per se, or, more preferably, to the clusters or sub-clusters. A geometrical model can be viewed as an operator, G, associated with a specific geometry, acts on a collection of picture-elements, and provides characteristics parameters and, optionally, the goodness of fit of the respective geometry to the collection.

When the geometrical modeling procedure is applied to clusters, it can be used to define sets of clusters. The spatially defined clusters serve as initial elements from which other sets are collected by internal criteria (one cluster set), external criteria (many cluster sets) or combination of internal and external criteria, as further detailed hereinunder.

A representative example of a geometrical model is a disk. The characteristics parameters for such geometry include the radius of the disk and the location of its center of mass. The goodness of fit can be expressed, for example, in terms of circularity. A disk model is particularly practicable for identifying, for example, blood cells. Once the characteristics parameters and goodness of fit for the disk model are obtained for a collection of cells, sifting can be performed based on various criteria including, without limitation, radius size, spatial location, circularity and the like.

Another example for a geometrical model includes the use of fractal geometry, in which case the characteristic parameter can be the dimension of lines in the image. Many types of dimensions are contemplated, including, without limitation, fractal dimension, Hausdorff dimension, correlation dimension, information dimension, Lyapunov dimension and Minkowski-Bouligand dimension. Line-dimensions are useful for analyzing and characterizing borderlines between two adjacent tissue regions of the specimen. For example, the fractal dimension of the dermal epidermal junction can be used to define the borderline of the rete ridges.

An additional example for geometric modeling includes the calculation of the gray scale principal axis of the entire specimen. This type of geometric modeling is applicable also for individual picture-elements without clustering. Gray scale principal axis is particularly useful for monochrome images because such calculation does not utilize spectral classification.

The clustering and geometrical modeling procedures can also be applied in combination, e.g., for the purpose of analyzing overlapping regions, in which it is difficult to uniquely identify clusters of picture-element due to of image data entanglement. For example, when the image includes overlapping circles there is a risk of double counting or under-counting in the overlapping region. In such cases, the clustering and geometrical modeling procedures are preferably combined with the requirement for a better statistical fit to the geometric model. As a representative example, consider overlapping DAPI stained cell nuclei. In this case, disk models for sub-clusters and a better statistical agreement of the fit when additional parameters (models that invoke more disks) are considered.

Once the sets of picture-elements are defined, they can be associated with tissue regions, which, in turn can be associated with tissue elements of the pathological specimen.

For example, assume that a standard α smooth muscle actin staining agent is applied to a heart tissue and a pixilated spectral image of the heart tissue is obtained. The agent typically stains blood vessels epithelial cells. However, in the staining procedure relics and contaminations of the staining agent are expected to appear on other tissue elements. In step 18, described above, all pixels stained with the staining agent are affiliated with the same classification group. This group includes both pixels representing epithelial cells and pixels representing contaminations of relic staining. In step 20, described above, the method defines one or more candidate sets of the stained pixels which are associated with the blood vessel. When the geometrical modeling procedure is employed, the candidate set includes only group of stained pixels having a specific geometry which is consistent with the shape of the tissue of interest. In the present example, the candidate sets can include group of stained pixels forming a shape of, say, a non-circular ring.

Results of higher confidence level can be obtained by isolating the candidate sets for which the fit to the geometrical model is of higher quality. Other criteria can also be applied to sharpen the set definition and to eliminate falsely identified set elements. The other criteria preferably include various parameters of the geometrical model, such as, but not limited to, size, thickness and the like.

The candidate sets can also be cross-correlated with all clusters of the background spectrum class (background light or counter stain). In the above example of pixilated spectral image of the heart tissue, the correlated set represents the lumen in the blood vessel section. The conjunction of the two sets forms the blood vessels set, the background which represents the potential lumen, and the diaminobenzidine stain which represents the vessel cells, in the inspected specimen.

Consider, for example, the set of all two-times magnification images of skin biopsy,

$${A}^{2} = {Y}_{n} {A}^{2 , n} .$$

The set of picture-elements, T, which corresponds to tissue region in the image, can be defined as:

$$T = {Y}_{i , j} \{ {a}_{i , j} \vert \{ ( {a}_{i , j} ∈ {A}^{2} ) ⋀ ( {a}_{i , j} ∉ {Y}_{k} {C}_{0 , k}^{2} ) \} \} , \qquad ( EQ . 6 )$$

where the classification group “0” corresponds to background light. As can be understood from the above equation, the definition T includes the condition that no background picture-element (belonging to any of the clusters C_{0,k}², k=1, 2, . . . ) is present in the tissue regions. T can be defined by imposing other or additional conditions. For example, in an alternative embodiment, particularly useful in cases in which no fluorescence is present, the definition of T includes the condition that the normalization factor for background pixels is the largest. In this embodiment, T is preferably defined as:

$$T = {Y}_{i , j} \{ {a}_{i , j} \vert \{ ( {a}_{i , j} ∈ {A}^{2} ) ⋀ ( {R}_{i , j} < {R}_{bg} ) \} \} , \qquad ( EQ . 7 )$$

where R_{bg} is the minimal value of the background normalization factor. Note that in principle, T is defined for a particular choice of the indices m and n. For clarity of presentation, m and n have been omitted from the above equation.

The tissue region of the image is provided by the method by means of a coordinate system. In the simplest case, the coordinate system is the Cartesian coordinate system of the pathological specimen. For example, when the pathological specimen is on a slide, the coordinate-system can be a two-dimensional Cartesian coordinate system with respect to an origin on the plane of the slide. For three-dimensional images, such as those provided by a con-focal microscope or using focus plane information to remove z-stacking degeneracy, the coordinate system can be a three-dimensional Cartesian coordinate system with respect to an origin on one of the planes of the image. Many other two- and three-dimensional coordinate systems are contemplated, as further detailed hereinunder.

According to a preferred embodiment of the present invention the method continues to step 22 in which one or more set-operators are applied on one or more sets or clusters of picture-elements. One of the main purposes of the set-operators is to characterize tissue regions in the specimen. Set-operators are mathematical entities. However, being applied to sets or clusters which are defined in accordance with image data of the pathological specimen, the set-operators can be associated with diagnoses describing the pathological specimen or portions thereof. Thus, according to a preferred embodiment of the present invention each set-operator is associated with a predetermined diagnosis describing the pathological specimen or a portion thereof. For example, a set-operator can be associated with a diagnosis criterion for determining malignancy of a tumor.

In practice, a list of set-operators and associated diagnoses is provided, and individual set-operators from the list can be applied on the sets or clusters. The outcome of the operation performed by each set-operator can then be interpreted in term of the respective associated diagnosis in the list. Based on results obtained by the application of the set-operator, a report describing the pathological specimen can then be issued. In most cases, the list of list of set-operators and associated diagnoses is provided from a library of set-operators.

Alternatively, step 22 can be preceded by an optional step 21 (which can be executed subsequently to any of the above method steps, except step 22) in which one or more set operators associated with known pathological diagnoses are defined.

The characterization of tissue regions by the set-operator(s) is preferably according to both image data and spatial characteristics of the set. The characterization of tissue regions can be done either statistically (e.g., by means of a density distribution) or deterministically (e.g., by means of a tissue scale).

A set-operator Ω, as used herein, refers to a mathematical transformation which uses the set (or cluster) S as an operand and transforms it to another representation F:
ΩoS→F (EQ. 8)

The set-operator Ω can perform the transformation either by treating the set S as a collection of elements and performing the transformation globally on the entire collection, or by transforming each individual element of the set. The set-operator can perform one or more mathematical operations on the set or cluster in order to provide the representation F. Any type of mathematical operation can be performed by the set-operator, including, without limitation, logical operations, algebraic operations, differential operations and integral operations. The representation F to which the set-operator Ω transforms the cluster or set S can be any type of mathematical representation, including, without limitation, a scalar (e.g., a statistical quantity), a pseudo-vector (e.g., a major axis), a vector (e.g., a list of statistical quantities, a preferred direction), a tensor (e.g., tensor of inertia), a matrix (e.g., a statistical correlation matrix), a function (e.g., a distribution function, correlation function), a set (e.g., a set of picture-elements which belong to a particular region of the image, a set of distribution moments) and the like.

In various exemplary embodiments of the invention the set-operator is used for calculating the coordinates of picture-elements or other objects (e.g., a set, a cluster, a sub-cluster) according to the desired coordinate system. The coordinate system can be a global coordinate system, where the entire specimen is described in terms of the same reference frame and relative to a single origin, or a plurality of local coordinate systems, where different objects (e.g., different clusters) are descried in terms of different reference frames and different origins.

A representative example of a global coordinate system is a coordinate system defined in terms of the principal axis of the tensor of inertia of the entire pathological specimen. Such global coordinate system is referred to herein as the “specimen coordinate system”. To calculate the coordinates in the specimen coordinate system the set-operator preferably calculates the tensor of inertia of the pathological specimen (e.g., via gray level or binary intensity thresholding) and expresses the location of various picture-elements in terms of the principal axes of the tensor. When the image is a two-dimensional image, the coordinate system is defined using two principal axes, p₁ and p₂; when the image is a three-dimensional image, the coordinate system is defined using three principal axes, p₁, p₂ and p₃.

A representative example of a local coordinate system is a coordinate system defined in terms of two or three principal axes of the tensor of inertia of an object belonging to a specific classification group. Such local coordinate system is referred to herein as the “object coordinate system”. To calculate the coordinates in the object coordinate system, the set-operator calculates the tensor of inertia of the individual object, and expresses the location of the picture-elements of the in terms of the individual object and optionally of one or more adjacent objects, in terms of the principal axes of the tensor. As will be appreciated by one ordinarily skilled in the art, the object coordinate system is local in the sense that the coordinates of different objects can be defined in terms of different individual coordinate systems.

The coordinate system can also be defined relative to a predetermined spatial location, region or a line on the specimen. The predetermined spatial location, region or a line can be selected to define coordinate system either locally or globally. For local definition, the coordinate systems of different objects are defined relative to different spatial locations, regions or lines on the specimen. For global definition, the coordinate system of all objects is defined relative to the same spatial location, region or line. A representative example of such global coordinate system is a coordinate system which is defined relative to the boundary of the tissue. In this embodiment the set-operator Ω measures the shortest distance ρ of each picture-element or object from the tissue's boundary, and assigns the measured distance to the respective picture-element or object. Such coordinate system is referred to herein as the “tissue boundary coordinate system”. For two-dimensional images, an angular coordinate θ (with respect, e.g., to the origin of the principal axis system) can be used as the complimentary coordinate and for three-dimensional images two additional coordinate (e.g., two angles, θ₁ and θ₂) can be used as the complimentary coordinates.

Beside the calculation of coordinates, the set-operator of the present embodiments can perform many other operations. According to a preferred embodiment of the present invention the set-operator is used in the definition of the sets T of picture-elements corresponding to tissue regions in the image. In the above example of ×2 magnification images A², Rᵢⱼ (see Equation 7) can be replaced by the cluster average normalization factor R̅_̅{̅s̅,̅k̅}̅, defined as:

$$\overline{{R}_{s , k}} = \frac{1}{{n}_{s , k}} \times {\sum }_{\{ ( i , j ) \vert {a}_{i , j} ∈ {C}_{s , k}^{2} \}} {R}_{i , j} , \qquad ( EQ . 9 )$$

where n_{s,k} is the number of pixels included in the kth cluster of classification group s. Thus, in this embodiment, set-operator Ω performs a cluster average over the normalization factors of the spectra and uses the cluster average for redefining the set T:

$$Ω oT -> {Y}_{i , j} \{ {a}_{i , j} \vert \{ ( {a}_{i , j} ∈ {C}_{s , k}^{2} ) ⋀ ( \overline{{R}_{s , k}} < {R}_{bg} ) \} \} , \qquad ( EQ . 10 )$$

the advantage of this redefinition of T is that it reduces or eliminates erroneous inclusion of outliers from both sides.

The set-operator of the present embodiments can also comprise mathematical operator or operators which performs any of the aforementioned mathematical operations to calculate numerous other quantities, including, without limitation, probability distributions, statistical distributions, statistical moments, correlation functions, distribution of parameters obtained from the geometrical modeling procedure, distribution moments (e.g., dipole, quadrupole or higher orders in a multipole expansion), set characteristics (e.g., dimension, area, density) and the like. The set-operator of the present embodiments can also performs various set operations including, without limitation, filtering (e.g., thresholding), smoothing (e.g., averaging with or without weights), separating overlapping sets, eroding, dilating, growing, shrinking and any combination thereof.

A representative example of an operation performed by the set-operator of the present embodiments is the calculation of various quantities which are related to tissue morphology. In this embodiment, Ω preferably calculates quantities which are related to the density of tissue in the pathological specimen. For example, Ω can calculate the ratio between the dimension of sets corresponding to specific tissue regions and the dimension of sets corresponding to other tissue regions. Such ratio can be considered as a filling factor. Sets corresponding to specific tissue regions can be any of the aforementioned sets, including, without limitation, classification groups, clusters, sub-clusters and the like. A set corresponding to other tissue regions can include, e.g., all picture-elements identified as tissue excluding background picture-elements. Thus, in various exemplary embodiments of the invention Ω can be used for obtaining (i) the fraction of tissue area occupied by cell nuclei, (ii) the average number of cell nuclei per unit area in the entire tissue or in a specific tissue region, leading to a density map, and/or (iii) spatial distribution of various elements in the specimen.

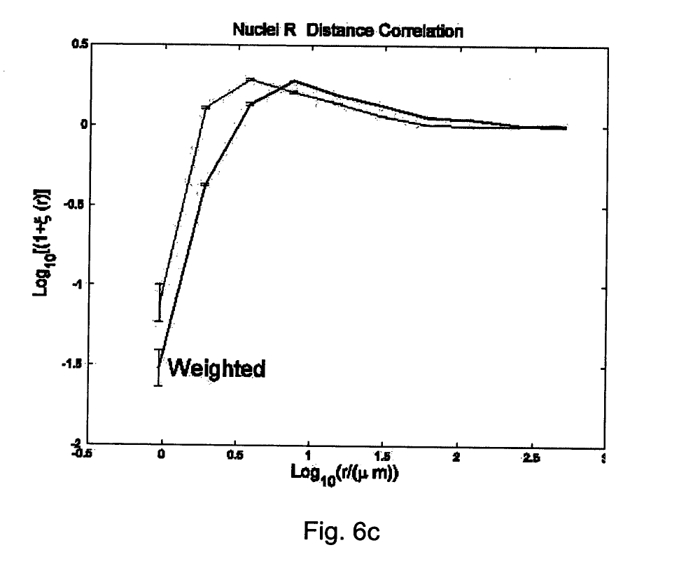

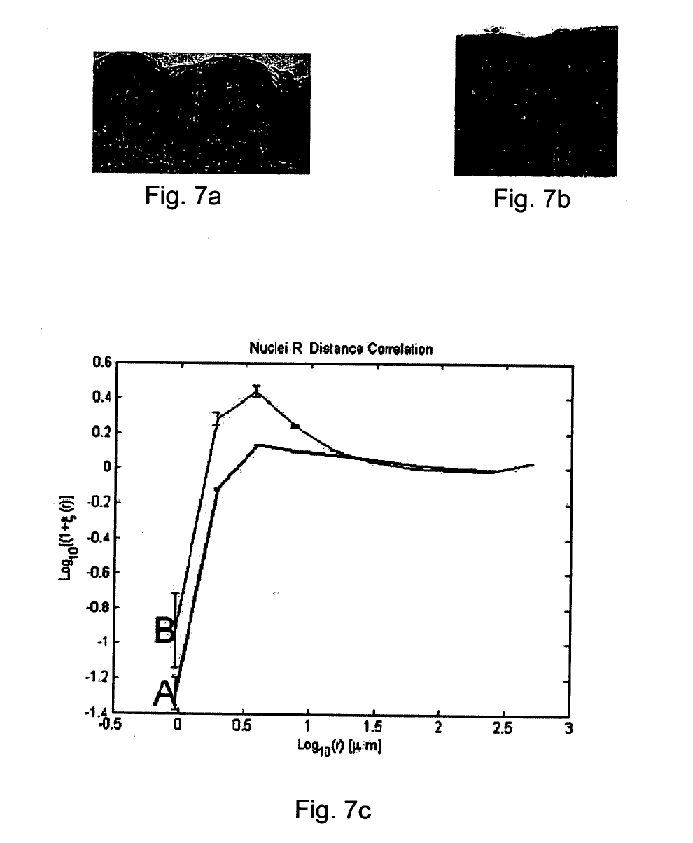

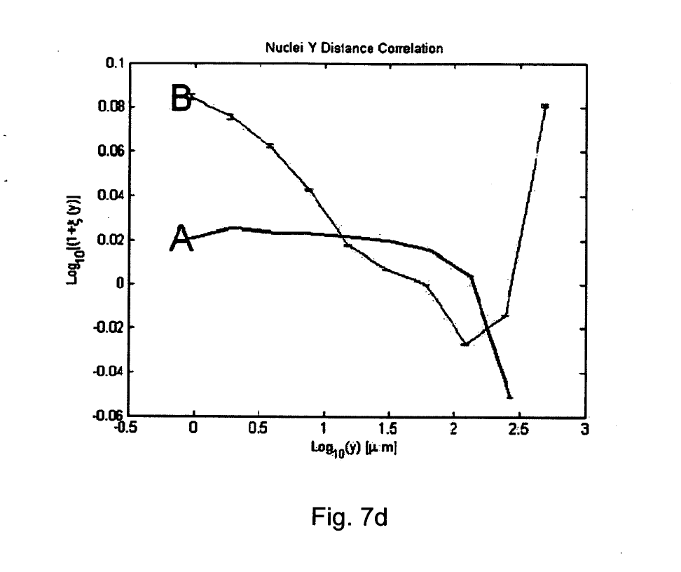

Another representative example of an operation performed by the set-operator of the present embodiments is the calculation of various probability distributions. For example, in one embodiment, Ω calculates a distribution which represents the probability of having a cluster of size n, where n can be measured in image units (e.g., pixels) or slide units (e.g., square microns). Such probability distribution is denoted herein by P(n). Representative example of P(n) is shown in FIG. 6a of the Examples section that follows.

In another embodiment, Ω calculates a distribution which represents the probability of that an area (or a volume in three-dimensional images) is populated by N clusters or populated picture-elements. The area or volume can be characterized by one or more parameters, thus making the distribution a function of one or more variables. For example, when an area in a two-dimensional image is characterized by one parameter R (which can be, e.g., a radius or a square) a single-variable probability distribution P_{N}(R) can be calculated for each population number N.

In an additional embodiment, Ω calculates a distribution which represents the probability of finding N clusters or populated picture-elements in a given area or volume. Similarly to the above, the area or volume can be characterized by one or more parameters. For example, when the area in a two-dimensional image is characterized by one parameter, R, a single-variable probability distribution P_{R}(N) can be calculated for each value of R.

In another embodiment, Ω calculates an n-point correlation function, ξₙ. For example, Ω can calculate a two-point distance correlation function ξ_{αβ}(r) defined as the excess (over random) probability of finding an object of type β at an absolute distance r from an object of type α, where the objects α and β represent a cluster, a sub-cluster or a populated picture-element. ξ_{αβ}(r) can be expressed in terms of an excess probability with respect to random distribution. The absolute distance r can be calculated on a two-dimensional plane. Alternatively, when a multi-layer object is detected, r can be calculated in a three-dimensional space. In the special case in which α=β, the function ξ_{αβ}(r) is the auto-correlation function. The absolute distance r can be measured between the center of mass of the objects or any other point in them. A correlation length, r₀, can also be defined to describe the length at which the (normalized) correlation equals 1. ξ_{αβ}(r) can also be a weighted distribution, in which case the sizes of the objects can be used as weights.

The distance correlation function ξ_{αβ}(r) described is not sensitive to the direction of the line connecting the two objects. The set-operator Ω can also calculate various quantities which are related to directional distributions.

Thus, according to a preferred embodiment of the present invention Ω calculates the correlation as a function of the coordinates of the objects α and β (as opposed to the absolute distance r). The type and number of coordinates depending on the selected coordinate system. For example, when a two-dimensional Cartesian coordinate system is employed (e.g., with respect to an origin on the slide's plane) the correlation functions include ξ_{αβ}(x) and ξ_{αβ}(y), which are calculated for the x coordinate and the y coordinate of the Cartesian coordinate system, respectively. When a three-dimensional Cartesian coordinate system is employed the correlation functions include ξ_{αβ}(x), ξ_{αβ}(y) and ξ_{αβ}(z) which are calculated for the x, y and z coordinates, respectively. When the specimen or object coordinate system are employed, the correlation functions include, ξ_{αβ}(p₁) and ξ_{αβ}(p₂), which are calculated for the first (p₁) and second (p₂) principal axes of the tensor of inertia. For three-dimensional images an additional correlation function ξ_{αβ}(p₃) can be calculated with respect to the third axis. When the tissue boundary coordinate system is employed, the correlation functions include ξ_{αβ}(ρ) and ξ_{αβ}(θ), which are calculated for the ρ coordinate and the corresponding angle θ defined above. For three-dimensional images two angular correlation functions ξ_{αβ}(θ₁) and ξ_{αβ}(θ₂) can be calculated with respect to two angular variables θ₁ and θ₂.

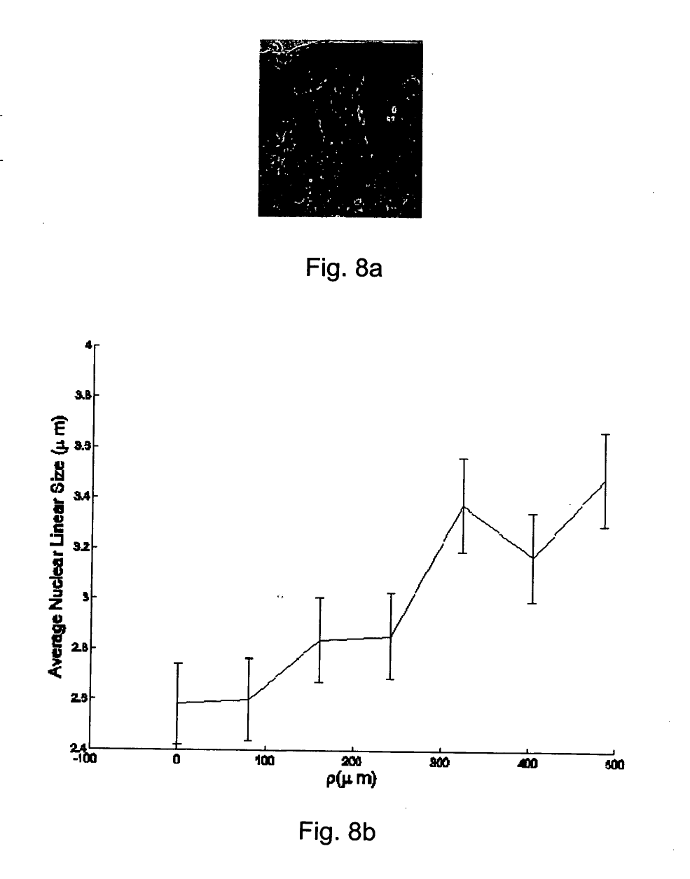

Ω can also calculate ratios between any two correlation functions. For example, Ω can calculate ratios, between a directional correlation function [e.g., ξ_{αβ}(x), ξ_{αβ}(y), ξ_{αβ}(p₁), ξ_{αβ}(p₂), ξ_{αβ}(ρ), ξ_{αβ}(θ)] and ξ_{αβ}(r), which, as stated, is not sensitive to different directions. Such ratio can serve as an indicator for the isotropy level of the tissue. Various calculations of correlation functions are demonstrated in FIGS. 6a-c and FIGS. 7a-d of the Examples section that follows.

In a further embodiment, Ω calculates the average number and/or size of objects per unit area as function of the tissue coordinate.

In yet another embodiment, Ω calculates spatial distribution cross-talk of one or more geometrical shapes. For example, Ω can calculate the probability Ψ(φ₁, φ₂; r) of a modeled object to have a position angle φ₂ of its long axis, given a modeled object with position angle φ₁, at a distance r. Ψ can be expressed in terms of an excess probability with respect to random distribution. Ω preferably calculates Ψ by averaging cos(φ₁−φ₂) over the sample pairs as a function of r. Ψ can be normalized, for example, with respect to random position angle distribution. Additionally, the calculation of Ψ can be done under various constraints, including, without limitation, correlation threshold, eccentricity threshold and the like. For example, in one embodiment Ω calculates a nest distribution, by first isolating only regions with high level of auto-correlation between cells, and then calculating the correlation between the “center of mass” of all such regions. Ω can also calculate cross-talks between the characteristics of a particular object and global features of the specimen. A representative example for such cross-talk is the average eccentricity as function of local nuclear density.

A particular feature of the present embodiments is the ability to identify rare clusters. As stated, clusters can correspond to cells, nuclei, blood vessels, connecting tissue or any other regional component in the specimen. Mathematically, a rare cell is a set derived by applying one or more set-operators to a set or a cluster. A rare cell can be identified in accordance with present embodiments of the present invention using the parameter distribution function of all the cells. For example, a cell can be defined as a “rare cell” if it has an eccentric shape. Such cell can be identified, e.g., by calculating the eccentricity distribution of all the clusters that represent cells and looking for an outlier within the eccentricity distribution. The eccentricity distribution can be calculated from any field-of-view, including, without limitation, the specific field-of-view under inspection, adjacent fields-of-view, the entire slide, the entire tissue (e.g., when different sections are spread over several slides) or an archive of modeled such cells.

Another particular feature of the present embodiments is the ability to identify abnormalities in the specimen. An object is referred to as “abnormal” if its distribution is significantly different from some known reference distribution for the same or similar object. For example, in solid tumors a distribution tail of the size or shape of nuclei can reflect the increased number of metastasized cells. In such cases, the comparison between the distribution of the size or shape of the nuclei, and a reference distribution can provide a reliable diagnostic. The advantageous of employing distribution comparison is that it reduces errors due to the sample finite size (number of detected nuclei of a specific type), the nuclear modeling and the like.

An additional representative example of an operation performed by the set-operator of the present embodiments is the calculation of quantities which characterize the global morphology or geometry of the pathological specimen. For example, Ω can calculate skewness about one or more principal axes of the global tensor of inertia (the tensor of inertia of the entire pathological specimen). Alternatively or additionally, Ω can compare between principal axes of different local tensors of inertia (tensors of inertia of objects). For example, Ω can calculate the angle φ_{αβ} between the primary principal axis of object α and the primary principal axis of object β. The same applies to principal axis of sets of objects.

An additional representative example of an operation performed by the set-operator of the present embodiments is the calculation of quantities which characterize distinct regions corresponding to different tissue types of the pathological specimen. For example, Ω can locate the dermis-epidermis junction line by calculating the average normalization factor across the image and finding the largest gradient line of the average normalization factor, which in turn can be associated with the dermis-epidermis junction line. The dermis-epidermis junction line can be used, for example, for comparing between the thicknesses of the epidermis and the dermis. Ω can also calculate the fractal behavior of distinct regions or lines. For example, in skin samples, Ω can calculate the fractal dimension of the dermis-epidermis junction line.

Ω can also calculate the average concentration of a specific probe (e.g., a fluorescent DNA probes such as Her2Neu), thereby to provide a smooth field of the specific probe.

Other representative examples of set-operators and their operations are provided in the Examples section that follows.

The present embodiments also contemplate combination with other technique, such as, but not limited to, counting techniques (e.g., FISH counting). This can be done combining information obtained from the counting technique with the information obtained using the set-operators. Thus, according to a preferred embodiment of the present invention the method continues to step 24 in which one or more counting technique is performed prior to or subsequently to any of the above method steps. The results of the counting technique can then be correlated with the sets of picture-elements or other representation obtained using the set-operators between the results of the two analyses can be calculated. For example, a correlation function can be calculated between FISH markers of the counting technique and sets of picture-elements corresponding to specific targeted cells. Positive or negative correlations between the two maps can be used as indicators for the accuracy of the analyses.

One example of such combination is the Her2Neu tests applied to patients with breast cancer. Thus, according to a preferred embodiment of the present invention the method is applied to a tumor slide for detecting Her2Neu expression and classifying the detected expression level. If the expression level is marginal or undetermined, a cytogenetics test is applied to the same tumor slide. In the cytogenetics test, the Her2Neu gene is preferably marked by FISH and the amplification level with respect to a control marker is measured as known in the art. Subsequently, a smoothed map of cell population type from the first analysis is compared to a smooth amplification map from the second test, where the two maps are preferably smoothed using the same smoothing scale. Regions on the slides in which correlation between the two maps is found can be interpreted as suspected regions, more than one oncogene, or the progress of the pathologic condition.

In various exemplary embodiments of the invention the method continues to step 26 in which a report describing the pathological specimen is issued. The report preferably comprises a quantitative diagnosis of the specimen or a portion thereof. The quantitative diagnosis is achieved in accordance with preferred embodiments of the present invention by the performing the mathematical operations as delineated above and further exemplified in the Examples section that follows. Thus, the definition of sets in the image and the application of set-operators on the sets provide quantitative and unbiased diagnosis to the pathological specimen.

The method ends at step 28.

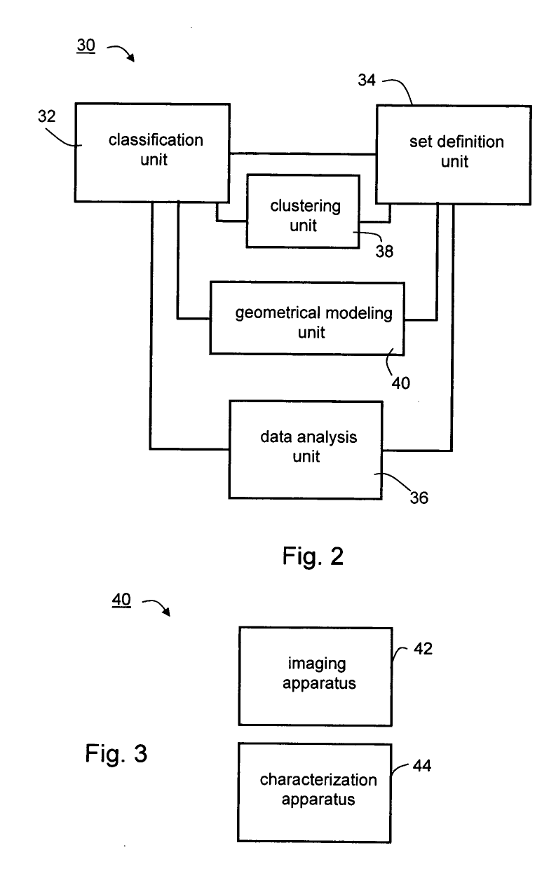

Reference is now made to FIG. 2, which is a schematic illustration of an apparatus 30 for characterizing a stained pathological specimen, according to various exemplary embodiments of the present invention.

Apparatus 30 can implement selected steps of the method illustrated in the flowchart diagram of FIG. 1 above.

Apparatus 30 can be commonly distributed to users on a distribution medium such as an electronically readable data storage medium in a form of computer programs. From the distribution medium, the computer programs can be copied to a hard disk or a similar intermediate storage medium. The computer programs can be run by loading the computer instructions either from their distribution medium or their intermediate storage medium into the execution memory of the computer, configuring the computer to act in accordance with the method of the present embodiments. All these operations are well-known to those skilled in the art of computer systems.

Apparatus 30 comprises a classification unit 32 which classifies picture-elements into classification groups according to image data, and a set definition unit 34 which uses the classification groups to define one or more sets of picture-elements, corresponding to tissue regions of the pathological specimen, as further detailed hereinabove. Apparatus 30 further comprises a data analysis unit 36 which applies the aforementioned set-operators the sets as further detailed hereinabove and in the Examples section that follows.

In various exemplary embodiments of the invention apparatus 30 comprises a clustering unit 38 which preferably communicates with classification unit 32 and set definition unit 34. Clustering unit 38 which clusters the picture-elements according to the classification groups. Clustering unit 38 can provide clusters as well as sub-clusters as further detailed hereinabove. Apparatus 30 can further comprise a geometrical modeling unit 40 which applies the aforementioned geometrical modeling procedure to picture-elements, clusters and/or sub-clusters as further detailed hereinabove.

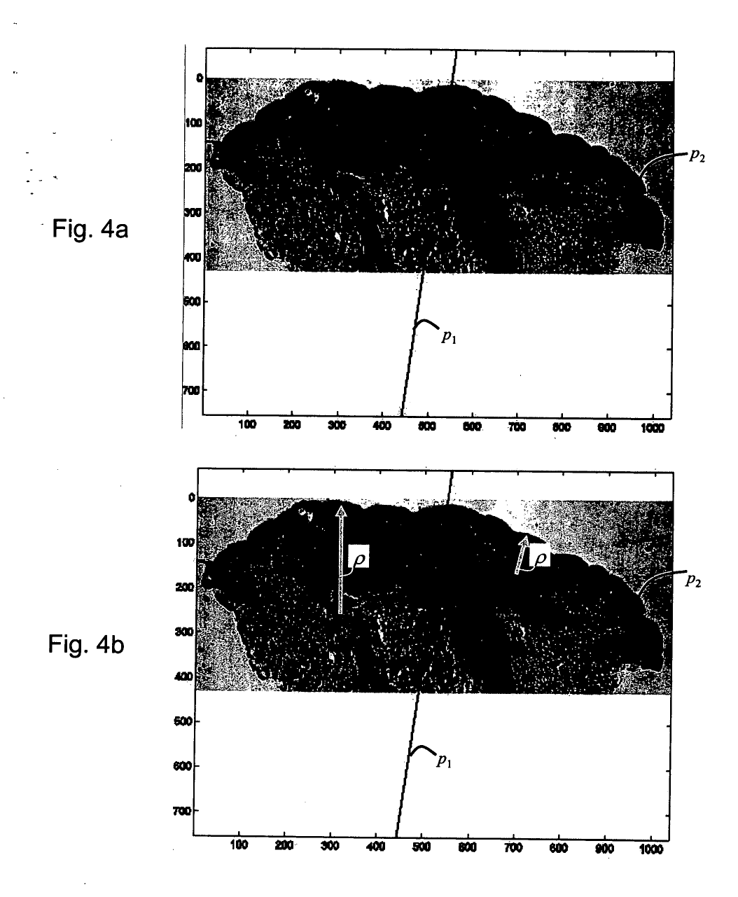

Reference is now made to FIG. 3, which is a schematic illustration of a system 40 for characterizing a stained pathological specimen, according to various exemplary embodiments of the present invention. System 40 preferably comprises an imaging apparatus 42 which provides the image of the specimen, and a characterization apparatus 44 which characterizes the specimen. Characterization apparatus 44 can be for example, apparatus 30.

The characterization of the pathological specimen according to preferred embodiments of the present invention, depends on the magnification and the resolution capabilities of the imaging apparatus, and on the size of the field provided thereby.

Imaging apparatus 42 typically includes means to acquire the image and it may also include means for magnifying the image (e.g., a microscope) and/or means for illuminating the specimen. Means for acquiring the image can comprise a charge-coupled device (CCD) which acquires the image by translating the light into electronic impulses and transmits the image to a display device. The illumination means can comprise a white light source or a wavelength specific light source (e.g., an ultraviolet light source and the like). Such means are well-known to those skilled in the art of imaging. Imaging apparatus 42 can provide monochrome images or color images, as desired. It is recognized that while color images are useful for fast imaging of a relatively small number of stains, monochrome images can be used for stained specimen with a large number of stains.

In various exemplary embodiments of the invention imaging apparatus 42 comprises a spectrometer which accepts light, separates it into its spectral components and measures the intensity of the light as a function of its wavelength.

Imaging apparatus 42 preferably comprises one or more filters which can be used in bright field or dark field imaging. In bright field imaging, the filters can operate in the visible light (e.g., a green filter, a brown filter, etc.). In dark field imaging the filters can be fluorescent filters, in which case they can include, without limitation, excitation filters and emission filters, as known in the art. The imaging apparatus can also comprise dichromatic beamsplitter (e.g., a mirror). The means for acquiring the image (e.g., CCD) is used in combination with the aforementioned filters, where a plurality of exposures of the CCD is performed, each time with a different filter, to provide a spectral image of the specimen.

Imaging apparatus 42 can also provide three-dimensional images. In this embodiment, imaging apparatus 42 can comprise a, for example, a con-focal microscope. Alternatively imaging apparatus 42 can employ focus plane information which removes z-stacking degeneracy.

It is expected that during the life of this patent many relevant imaging techniques will be developed and the scope of the term imaging apparatus is intended to include all such new technologies a priori.

Additional objects, advantages and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions illustrate the invention in a non limiting fashion.

### Example 1

### Calculation of Coordinates

In various exemplary embodiments of the invention the set-operator Ω performs at least one operation for calculating coordinates according to the selected coordinate system. Ω can be used for calculating any coordinate system, including, without limitation, the object coordinate system and the tissue boundary coordinate system. In various exemplary embodiments of the invention Ω performs one or more of the following operations:

(i) For a specific classification group G in the set T, Ω can calculate the tensor of inertia from all the picture-elements in the group. Alternatively, Ω can collapse all the clusters in G to points (e.g., center of mass points) and calculates the tensor of inertia, I, from the collapsed clusters with no regard to the cluster size. The principal axes p₁ and p₂ of the tensor I, can then define the object coordinate system.

(ii) Ω can locate the connecting line between “upper” skin epidermis layer (stratum corneum) and picture-elements belonging to the background light classification group. A representative example of a procedure for identifying the epidermis layer is provided in Example 6 below.

(iii) Ω can measure the minimal distance of every point to the connecting line and defines it to be the “ρ coordinate” of the picture-element.

(iv) Ω can measure the angle θ of the connecting line relative to the primary axis of the tensor of inertia and the center of mass. This angle can be used as the complementary coordinate to ρ.

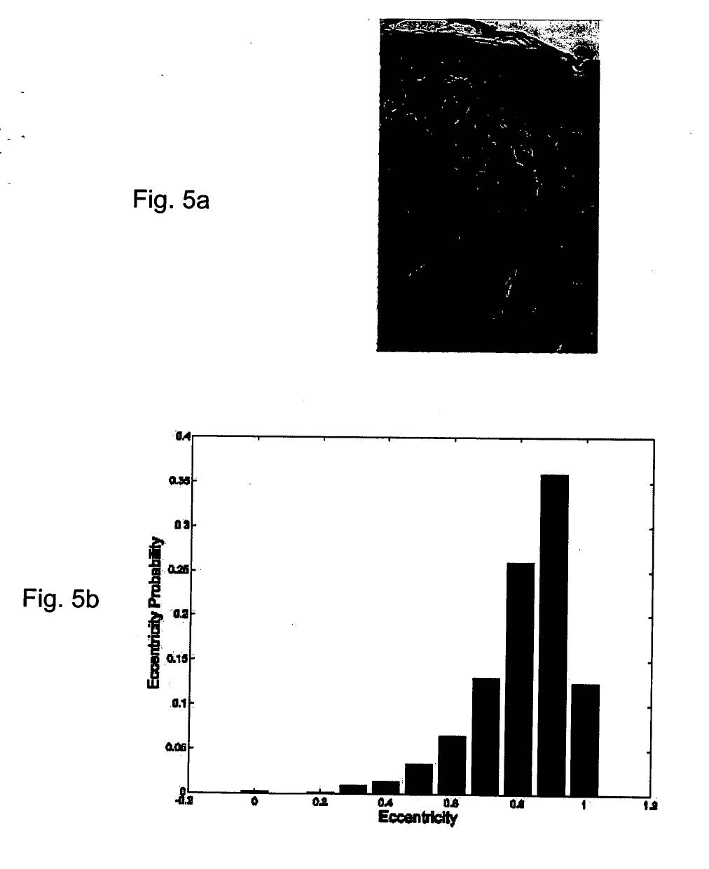

FIG. 4a shows Hematoxylin and Eosin staining of skin melanocytic nevus section which is superimposed by its principal axes p₁ and p₂. The axes are calculated from a subset (H) of picture-elements which bear spectral resemblance to the hematoxylin spectrum. The coordinates on the margin of the image correspond to the coordinate system of the slide (pixel enumeration, in the present example). The axes intercept at the center of mass of the H subset.

FIG. 4b shows a skin lesion section of FIG. 4a with two representative examples of the ρ coordinate indicating the distance from the upper edge of the upper skin epidermis layer (stratum corneum).

### Example 2

### Tissue Characteristics

In the present example, the set-operator Ω calculates the parameter distribution of specific population. The parameters are taken from a geometrical modeling of the set, which, in the present example is taken to be an ellipse. Ω can express the parameter distribution in terms of a probability distribution function (PDF). Additionally, Ω can calculate several distribution moments, such as, but not limited to, average, variance, skewness, kurtosis and the like.

Following is a description of a procedure for characterizing cell nuclei and/or nucleoli in a tissue section.

Definition of the Set

The set T of picture-elements which corresponds to tissue region is preferably defined using one or more of the following steps:

(i) Identify nuclei picture-element candidates by their association with their classification group.

(ii) Cluster the identified nuclei candidates.

(iii) Apply a minimal cluster size cutoff to eliminate noise, such as the noise resulting from stain relics.

(iv) Perform initial geometrical modeling by fitting the shape of the clusters to an ellipse having a major axis a and a minor axis b. Characterize the geometrical model by the ellipse parameters (a and b), the position angle (φ), and the goodness of the fit (GoF).

(v) Perform sub-clustering to eliminate projection overlaps between nuclei and limited spectral-spatial resolution. Perform an additional geometric modeling, as in the initial geometrical modeling, but for the sub-clusters.

In some cases, the order of execution of steps (i) and (ii) above can be reversed, namely, clustering followed by spectral classification.

Set-Operator

The set-operator Ω can perform any of the following operations on T, once T is defined:

(i) Ω can calculate the statistical distribution of any of the parameters of the ellipse, including the major axis a, the a minor axis b, and the eccentricity ε of the ellipse, defined by ε²=1−b²/a². The statistical distribution can be a simple distribution or a weighted distribution. When Ω calculates a weighted distribution, various functions of GoF can be used as weights. Ω can calculate correlation matrix between GoF and any of the ellipse parameters.

(ii) Ω can calculate distribution of the spectral normalization factors Rᵢⱼ^{n,m} of T within each identified nucleus.

(iii) Ω can also perform sub-clustering by the values Rᵢⱼ for the purpose of nucleoli (sub-element) identification.

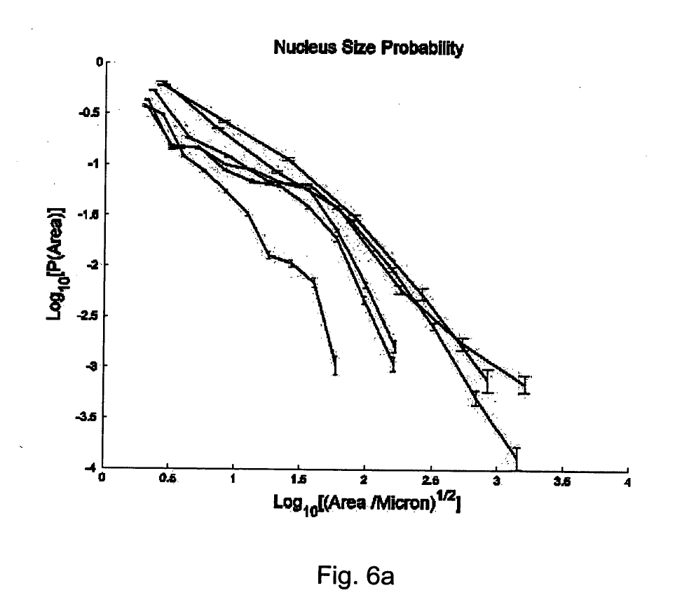

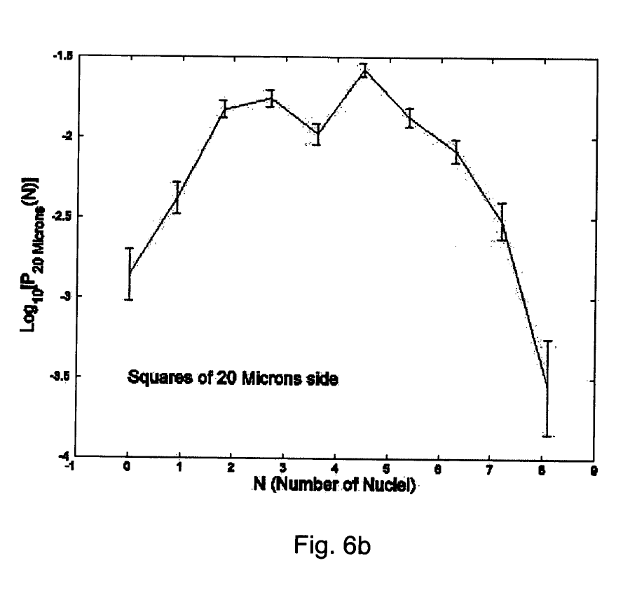

FIG. 5a shows cell nuclei field (red) superimposed over the color image of a skin lesion section, stained by Hematoxylin and Eosin. This demonstrates the spectral classification power and then followed by segmentation, labeling, modeling and the rest of the particular analysis steps.

FIG. 5b is a histogram showing the probability distribution of the nucleus eccentricity of the image of FIG. 5a after these underwent the ellipse modeling procedure.

### Example 3

### Tissue Morphology

In the present example, the set-operator Ω calculates various quantities which are related to tissue morphology. The following description is for the case of cell nuclei in a tissue section. The set T can therefore be prepared by executing selected steps of the procedure described in Example 2 above.

As stated, Ω can calculate various probability distributions. FIG. 6a shows a representative example of P(n) which represents the probability of having a cluster (nucleus) of size n. Shown in FIG. 6a is probability distribution for clusters defining nuclei as a function of the base-10 logarithm of the size of the nuclei measured in square microns. The probability distribution was calculated for 5 different skin lesions under ×20 magnification. Similar logarithmic slope can be observed for nuclei of linear size up to about 6 microns (1.5 in the logarithmic scale). One prominent outlier lesion was observed.

FIG. 6b shows a representative example of P_{R}(N) which, in this example, represents the probability of finding N clusters or populated picture-elements in an area enclosed by a square whose linear dimension is R. Shown in FIG. 6b is P_{R}(N) for a R=20 microns square. The probability distribution was calculated for one skin lesion under ×20 magnification.

FIG. 6c shows a representative example of the distance correlation function ξ_{αβ}(r), defined as the (excess) probably of finding an object of type β at an absolute distance r from a reference object of type α. In the present case the auto-correlation function, ξ_{αα}(r) is calculated. The ξ_{αα}(r) of was calculated for a skin lesion under ×20 magnification. Shown in FIG. 6c is a weighted by cluster size (blue) and un-weighted (red) distance correlation function.

Other calculations of correlation functions also are demonstrated in FIGS. 7a-d, showing color images of two skin lesion sections (FIGS. a-b), ξ_{αα}(r) (FIG. 7c) and ξ_{αα}(ρ) (FIG. 7d). ξ_{αα}(r) and ξ_{αα}(ρ) were calculated for each of the two skin lesion sections. In FIGS. 7c-d, the correlation function for the skin lesion section of FIG. 7a is designated by “A”, and the correlation function for the skin lesion section of FIG. 7b is designated by “B”.

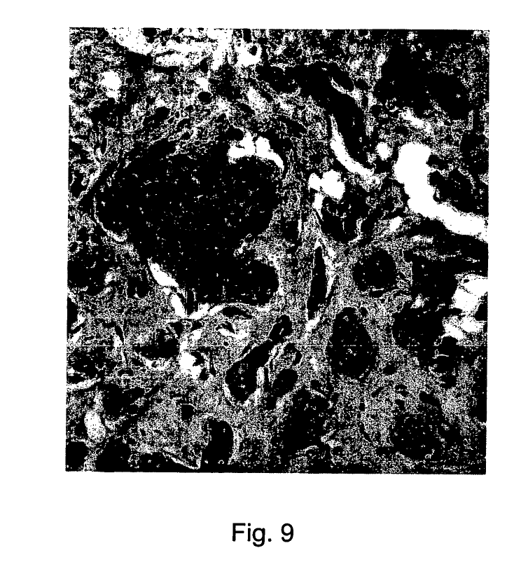

FIGS. 8a-b demonstrate the calculation of average size of nuclei. Shown in FIGS. 8a-b are skin lesion section (FIG. 8a) and the average linear size of nuclei as function of their distance ρ from the upper edge of the section (FIG. 8b).

### Example 4

### Calculation of Expression Levels

It is recognized that expression levels of markers (such as Her2/Neu, Estrogen, Progesterone, p53, etc.) are oftentimes misleading in the sense that the concentration (also referred to in the literature as “amplification”) of the markers are functions of the location in the tissue. Furthermore, in immunohistochemical specimen there often exist a few “expression levels” that correspond to different concentrations of the antibody in the suspected cells. Expression levels are traditionally judged by the distribution of the cells (within or outside of cell “nests” or “clusters”, i.e., tumor cells or not) average intensity. Many times, however, only cells that belong to a specific classification group or shape set are taken into account. The judgment of an expression level of a single cell is obtained by eye assessment, which is far from being accurate.

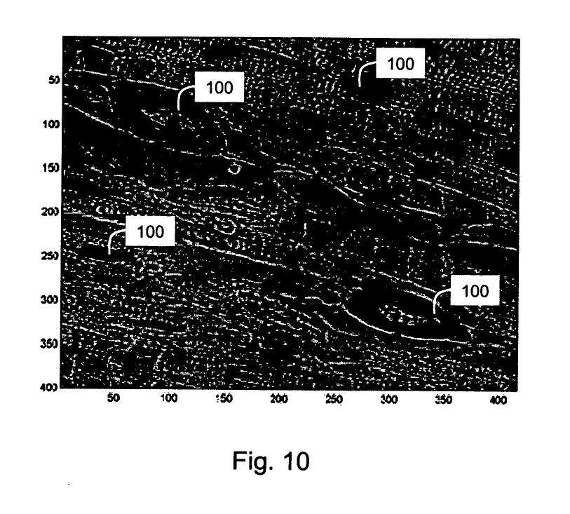

FIG. 9 shows a suspected tissue biopsy taken from a woman's breast and stained with ki-67 staining. Ki-67 gives a strong indication for proliferation and metastasis. Prior art pathology techniques quote two results based on visual immunohistochemical inspection: (a) the percentage of malignant cells with any ki-67 expression level out of the total number of malignant cells (defined by morphology or location); and (b) a scale of 0-3+ of expression levels. The scale typically represents the expression level within a particular cell (the signal intensity). It is then commonly fed into the following formula to provide the so called “H score”:

$$H = 100 {\sum }_{Levels} {E}_{i} {f}_{i} , \qquad ( EQ . 11 )$$

were the summation is over the expression levels, Eᵢ, times the fraction fᵢ of cells that show expression level Eᵢ.

The present embodiments successfully provide a procedure for calculating expression levels. According to a preferred embodiment of the present invention the procedure begins by classifying the pixels of the image corresponding to pure Hematoxylin, and Hematoxylin+DAB (diaminobenzidine)—the dye usually used for staining Ki-67. The resulting classification includes four different groups according to the relative weights of the two ingredients in the super-composed spectrum. In the case of Ki-67 and Hematoxylin as presented in FIG. 9, the Ki-67 dominates by far over the Hematoxylin, even when the lowest expression level is considered. Hence, in this particular example, only a single classification group of Ki-67 is established.

The procedure continues by performing clustering separately on the two classification groups to provide a set C₁ corresponding to the Hematoxylin-defined cell nucleus candidates and a set C₂ corresponding to the Ki-67 defined cell nucleus candidates. The sets C₁ and C₂ are defined as:

$$\begin{matrix}{C}_{1} = {Y}_{k = 1}^{{N}_{1}} {C}_{1 , k} & and & {C}_{2} = {Y}_{k = 1}^{{N}_{2}} {C}_{2 , k} ,\end{matrix} \qquad ( EQ . 12 )$$

where Nₗ is the number of clusters of classification group l (the magnification index is omitted for clarity of presentation).

Optionally and preferably, the procedure continues by applying, to each sub-cluster, C_{l,k} a set-operator Ωₒₗ which separates overlapping cells:
ΩₒₗoC_{l,k}→(C_{l,k}_{₁}, . . . , C_{l,k}_{ₙ}), (EQ. 13)
to provide a new set of sub-clusters C_{l,k}_{₁}, . . . , C_{l,k}_{ₙ}.

The procedure continues by applying two operators, Ω_{size} and Ω_{Model}^{Gof}, to each cluster or sub-cluster to thereby define Hematoxylin cells CN₁ and Ki-67 cells CN₂, namely cells that show DAB spectral signature in them. CN₁ are preferably defined as follows:

$$Hematoxylin\_Cells = {CN}_{1} = {Y}_{k = 1}^{{N}_{1}} \{ {C}_{1 , k} \vert [ ( {A}_{min} < ( {Ω}_{Size} o {C}_{1 , k} ) < {A}_{max} ) ⋀ ( ( {Ω}_{Model}^{GoF} o {C}_{1 , k} ) > {GoF}_{min} ) ] \} , \qquad ( EQ . 14 )$$

where Ω_{size} measures the cluster or sub-cluster by pixel count translated to physical area through the magnification scale, and Ω_{Model}^{GoF} provides the GoF of the particular cluster to the invoked geometric model (e.g., an ellipse model). The parameters A_{min/max} are determined by a known nuclei size distribution, while the parameter GoFₘᵢₙ is determined to include all deformed (usually malignant) nuclei but to eliminate residual dyes and other noise sources.

CN₂ are preferably divided into “expression level sets” by applying an additional operator, Ω_{ex.l}, to each set element, to provide the average normalization factor of the element:

$${Ω}_{ex . l .} {o ( {CN}_{2} )}^{j} = {\overline{R}}_{{( {CN}_{2} )}^{j}} . \qquad ( EQ . 15 )$$

Alternatively, the different expression level sets of clusters can be predefined based on spectral segmentation.

The division into expression level sets can be performed either automatically using the PDF of R̅ values, or through predefined bin limits. The division process, according to the presently preferred embodiment of the invention results in no more than 4 sets of cells corresponding to different expression levels.

The procedure continues by applying morphology segregation to the identified cells, preferably by both GoF values of the geometrical model and by the values of the model derived parameters. An additional operator is applied to provide a smoothed density map of cells on a typical small nest scale (abut 100 μm in the exemplified image of FIG. 9.)

In an additional step of the procedure, cells are selected by applying operators which pass the sets through a predetermined set of criteria, which may be in the form of a simple threshold (e.g., local smoothed density threshold to verify nest environment) or in the form of one morphological tests (e.g., maximal eccentricity). These operators validate that only relevant cells remain for the final analysis. The sought after diagnostics parameters, (i) Ki-67 expression level and/or (ii) the Ki-67 H-score are readily calculated from the obtained cells as known in the art.

In the example presented in FIG. 9, the Ki-67 expression level of nested cells is 0.053±0.005 by number and 0.037±0.003 by area. The statistics was calculated for all nuclei in the range of Nucleus>(4.3)² square microns. Since almost all expressed cells have the highest expression level (3+), the H-score equals about 160.

### Example 5

### Identification and Characterization of Blood Vessels

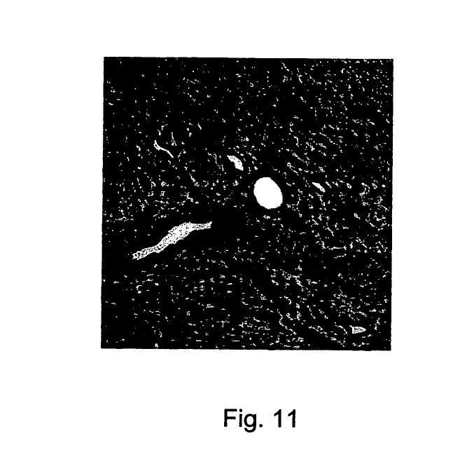

FIG. 10 shows a rat heart tissue section stained for epithelial cell identification (DAB-brown) with Hematoxylin counterstain. The lumen is seen as a “window” through which the background bright-field light (or counter-stain) appears. Also shown are model ellipses 100 for candidate vessels. Relics are removed from the image on the basis of size and poor model matching.

In accordance with various exemplary embodiments of the present invention, two types of clusters can be used to define a blood vessel. A first such type of clusters includes solid clusters (e.g., a disk, an oval, a blob) of epithelial cells. This type is particularly applicable in situations in which here (a) the vessel has collapsed during the specimen preparation; (b) a grazing cut of the vessel left no tracers of the lumen; or (c) the magnification level is too small for lumen resolution. A second type of clusters includes empty clusters (generally, but not obligatorily, non-circular ring) where epithelial cells encircle a cluster of background spectrum pixels (lumen). The second type of clusters can also include empty clusters with debris inside.

The preferred characterization procedure of the pathological specimen of the present example therefore includes a geometrical modeling step for the purpose of identifying the two types of clusters. The geometrical modeling procedure attempts to fit each cluster to a solid figure, an empty figure or an empty figure with debris. The geometrical modeling also provides the dimensions of each vessel and its lumen, their classified shape and the corresponding modeling errors.

Once the geometrical modeling is completed, all vessels can be put on a single reference basis, either on the same examined slide or on comparable slides.

The Blood Vessel Density (BVD) is a two-dimensional measure, derived from the section of a three dimensional-distribution. It is recognized that the derivation of a two-dimensional measure from a three-dimensional distribution involves a hidden assumption of random section direction. Specifically, denoting the angle between the vessel plane (perpendicular to the blood flow direction) and the section plane by θ, the random section direction assumption allows one to proceed and assume that the underlying cos(θ) distribution in different sections is identical and therefore comparison between different sections is statistically valid. The BVD measure, therefore, does not reflect the true three-dimensional vessel density, but rather its two-dimensional projection. Nonetheless, the random section direction assumption is corroborated by appealing to the vessel shape distribution.

Alternatively, one assumes a primary circular shape of the vessel sections, and transforms the vessel measured shape to a circular shape by affined deformation transformations to obtain a reconstructed primary vessel distribution. However, unlike random direction assumption, this procedure cannot be applied when grazing sections are involved. Yet, the probability for grazing sections is known to be rather low.

In various exemplary embodiments of the invention a blood vessel cluster, BV, is defined according to the following equation:

$$BV = {C}_{s , k} \vert \{ \begin{matrix}\{ min s < {C}_{s , k} < max s \} ⋀ \{ {Ω}_{GoF} o \{ {Ω}_{ring} o {C}_{s , k} \} > {min}_{GoF} \} ⋀ \\ ∃ {C}_{t} \vert \{ \{ {C}_{t} ∈ Lumen \} ⋀ \vert {Ω}_{c . m .} o {C}_{s , k} - {Ω}_{c . m .} o {C}_{t} \vert < {d}_{min} \}\end{matrix} \} \qquad ( EQ . 16 )$$

where, min s and max s are the minimal and maximal size of the (modeled or original) clusters that belong to the blood vessel spectral group, Ω_{GoF} is the Goodness of Fit operator of the ring model operator, Ω_{ring}, Lumen is a cluster of picture-elements which belong to the background spectral class and are surrounded by picture-elements of other spectral classes (namely not outside of the specimen boundaries), Ω_{c.m.} is the center of mass operator and dₘᵢₙ is a minimal distance between center of mass of two clusters. This definition of blood vessel can be modified, for example, by considering collapsed blood vessels having a reduced or no lumen therein. Such blood vessels can also contribute to the BVD calculation. In this case, the cluster BV is preferably combined with one or more clusters with smaller or no Lumen clusters.

Using the above or similar definition for the blood vessel cluster, the BVD can be characterized by applying one or more set-operators on BV.

The characterization of the pathological specimen according to the present example, preferably comprises many types of information. The upper and lower bounds for blood vessel size can be measured in a specific acquired field, utilizing a set-operator which calculates the actual cluster area (pixel summation) or the modeled cluster area, utilizing a specific geometrical model.

Additionally or alternatively, the vessel size distribution, e.g., the distribution of epithelial cell clusters, can be obtained utilizing one or more set-operators calculating distribution functions. Such set operators can also be applied on the Lumen cluster hence to provide the vessel projected cross section distribution.

Set-operators calculating distribution functions can additionally or alternatively be applied on the geometrical model to provide the vessel shape distribution. In such cases, the set-operators can provide, for example, the average wall thickness of the blood vessel or any other geometrical property thereof.

Another type of information is the blood vessel density, which can be obtained by applying a set-operator which calculates the number of vessels per unit area, or the number density of vessels of a particular size and/or shape.

The above distributions and densities can be accompanied by various derived quantities, including, without limitation, total cross section, cross section density, total number of vessels, average vessel size and various distribution moments. Optionally and preferably, one or more of the derived quantities are calculated as function of magnification, so as to obtain upper and lower bounds to the respective quantity or quantities. Selected sets of the blood vessels can be considered as a group to which spatial statistical operators can be applied and various statistics can thus be obtained and analyzed.

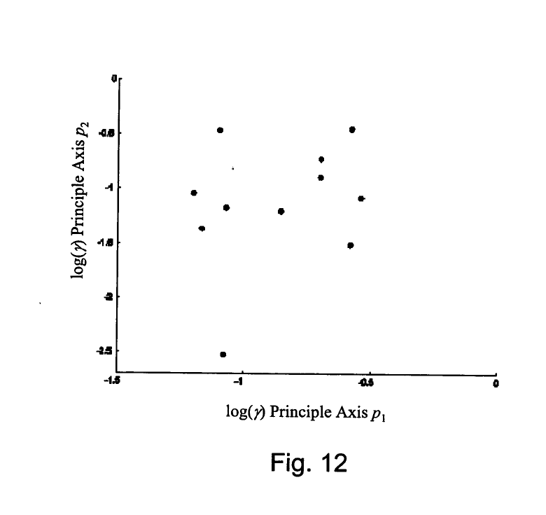

FIG. 11 shows BVD analysis for a tumor tissue. Shown in FIG. 11 are 22 vessels of sizes ranging between about 13.6² and about 130² square microns. The central hole should not be confused with a vessel, because it does not comply with the above definition of BV. Specifically, although the central hole comprises picture-elements belonging to the background spectral class, it is not a Lumen cluster, because it is not surrounded by picture-elements corresponding to the spectral class of blood vessel tissue. Yet, as stated, a few collapsed vessels clusters comprising a smaller or no Lumen cluster can be included.

The calculated blood vessel density in this example is about 2.4×10⁻⁵ (μm)⁻², the total cross section is about 5446 square microns and the cross section fraction in the tissue is about 5.9×10⁻³. The density was calculated by the counting the vessels defined previously and the information regarding the tissue size (number of pixels, magnification level and scale). The total cross section was calculated in two ways: (i) summation of lumen area within each vessel, and (ii) modeled area by applying the conjunction of an ellipse model for the lumen and a ring model for the vessel.

It is demonstrated that the BVD analysis performed according to a preferred embodiment of the present invention can be used for characterizing tumors growth accompanied by angiogenesis. The BVD analysis of present embodiments can therefore serve as a tumor precursor or phase estimator.

### Example 6

### Characterization of Skin Lesions

The present example demonstrates the ability of the present embodiments to characterize skin lesion sections. As is demonstrated below, the analytical approach devised by the present Inventor establishes a standard quantitative basis for many diagnoses and significantly reduces the amount of variations in the diagnosis.

Without limiting the scope of the present invention, the present example relates to the diagnosis criteria for malignant melanoma (MM). It will be appreciated that many of the techniques provided in the present example are applicable for other skin diseases or disorders, and one of ordinary skill in the art, provided with the details described herein would know how to adjust the procedure in accordance with the type of pathological specimen. Furthermore, selected steps of the procedure described in the present example can be used in the analysis of all Hematoxylin & Eosin stained specimen to analyze the architecture and morphology of the latter as well as other architecture-oriented stainings.

Known scales for grading MM include the Clark invasion level [Clark, W. H. J., 1969, “The histogenesis and biological behavior of primary human malignant melanoma of the skin,” in “Cancer research”, 29: 705-26; Ed. Bernardino, E. A., Mihm, M. C.], and the Breslow thickness [Breslow, A., 1970, “Thickness, cross-sectional area and depth of invasion in the prognosis of cutaneous melanoma,” Ann. Surg. 172: 902-908; and Breslow, A., 1975, “Tumor thickness, level of invasion and node dissection in stage I cutaneous melanoma” Ann. Surg. 182: 572-575]. It is recognized, however, that these two scales are not accurately defined. In particular there is no commonly accepted standard how to define and measure the invasion depth.

Following is a description of a preferred method for characterizing MM, devoid the above limitations. The analysis in the present example is applied in accordance with various exemplary embodiments of the present invention to spectral pixilated images like those shown in FIGS. 4a-b.

Symmetry

According to a preferred embodiment of the present invention, once the pixels are classified into spectral classification groups and the set are defined, one ore more set-operator are applied to determine the symmetry of the nevus. The assessment of the nevus symmetry is known to be useful for differentiation of MM from benign nevi [Cook et al., 1997, “The evaluation of diagnostic and prognostic criteria and the terminology of thin cutaneous malignant melanoma by the CRC Melanoma Pathology Panel,” Histopathology 30(2): 195-197].

Preferably, but not obligatorily, the determination of skin nevi symmetry is performed using a low magnification level that allows the scan of the entire section at low resolution. At low magnification level, where tissue elements are hard to be identified, spectra can still serve as good differentiators between the various tissue element concentrations.

The symmetry is defined with respect to the selected global or local coordinate system of the specimen. In various exemplary embodiments of the invention, the symmetry is determined using a plurality of local coordinate systems, such as, but not limited to, the object coordinate systems described above. Thus, according to the presently preferred embodiment of the invention the principal axes for the unification of several or all the clusters in several or all the spectral classification groups (excluding the background) are calculated, to provide a set of principal axes. The calculation can be performed by a simple summation (e.g., each nucleus is counted once), or more preferably, as weighted sums. In the latter case cluster sizes can be used as weights. The principal axes in the set can be compared in terms of their direction and magnitude to assess the nevus symmetry level.

In an alternative embodiment, the coordinate system is defined relative to a predetermined spatial location, region or a line on the specimen. Representative examples for such geometrical features include, without limitation, the tissue boundary, the dermis-epidermis junction line, etc. For example, the equidistance lines from the stratum corneum can be defined as the ρ coordinate and the perpendicular lines as the complementary coordinate y′. Alternatively, the complementary coordinate can be the angle θ between the ρ coordinate and the e.g., minor principal axis.

In any event, the set-operator preferably calculates one or more symmetry estimators in the framework of the selected coordinate system. Preferred symmetry estimators include, without limitation, parity, moments (e.g., skewness or higher order moments), and the like.

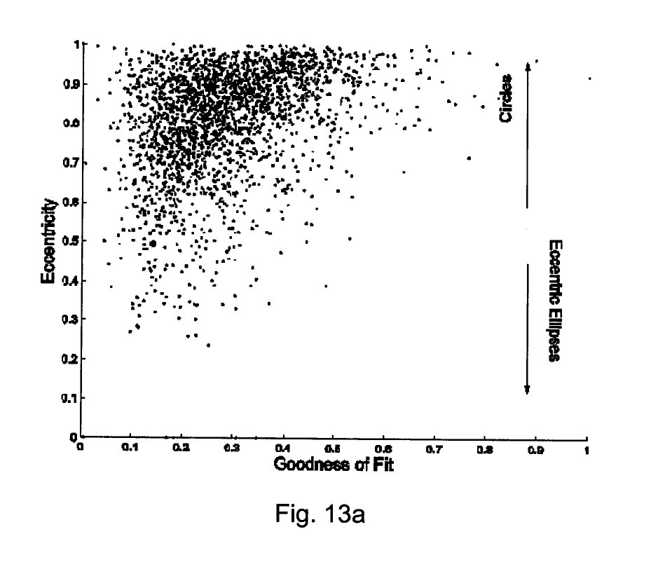

FIG. 12 shows a typical skewness distribution γ for skin lesion sections under low (×2) magnification. The skewness γ is calculated for a set of pixels that belong to the classification group of strong Hematoxylin spectrum. The skewness was calculated about the principal axes p₁ and p₂ (see FIGS. 4a-b). The skewness was defined as the distribution's third moment normalized by the standard deviation cubed. As shown, the skewness about p₁ is higher than the skewness about p₂. This is well understood since “up and down” symmetry (where “up” is toward the epidermis and the tissue edge) is not expected on the first place but rather serves for checking the sample symmetry features.

Grading

As stated, common scales for grading MM include the Clark level of invasion and the Breslow thickness. While Clark level refers to the degree of tumor invasion in terms of the deepest skin layer, the Breslow thickness is quoted in units of length (millimeters). Following are several examples for grading MM, in accordance with various exemplary embodiments of the present invention.

MM is known to be formed by non-controlled proliferation of melanocytes, which is accompanied by immune system reaction (presence of lymphocytes) and ulceration. Within the epidermis, where the melanocytes are surrounded by keratinocytes, one of the outcomes of the preparation (fixation) of pathological specimen is the destruction (e.g., by alcohol) of the melanocyte plasma skeleton fibers, while the cell membrane remains intact due to its environment. The appearance of melanocytes at the epidermis is hence a nucleus surrounded by background light. This appearance is referred to as “clefts”.

According to a preferred embodiment of the present invention the MM is graded by the identification of metastizing melanocytes. The melanocytes can be identified according to the teachings of the present embodiments by detecting clefts on the image. The procedure is similar to the procedure of blood vessels identification as further detailed herein above (see, e.g., Example 5). The difference between the detection of blood vessels and the detecting of clefts is that in the former case tissue regions are identified while in the latter case background regions are identified. The mathematical procedure, nonetheless, is the same.

Thus, a geometrical modeling procedure is applied for the purpose of identifying clusters of background pixels to be identified with clefts that at least partially surround clusters of hematoxylin-type (i.e., nuclei) pixels. The geometrical modeling also provides the dimension and shape of each cleft, and the corresponding modeling errors.

Metastizing melanocytes which are not surrounded by keratinocytes, can be identified by detecting clusters of pixels, which are representative of Metastizing melanocytes. For example, the stained nuclear hue of melanocytes is known to be darker than other tissue cell nuclei and bigger in size than the dark lymphocyte nuclei. Thus, according to a preferred embodiment of the present invention relatively large clusters of darker nuclear hue are identified as melanocytes candidates.

Once the melanocytes are identified, they can be classified into their cytologic types (10 such types are presently known) acceding to the size distribution and morphology of each identified cell or nucleus. Malignant melanocytes are preferably identified in cell or nucleus having a relatively large size and a characteristic morphology which can be spindle, dendritic, balloon or multinucleate morphology.

MM may appear similar to other nevi, such as the Spitz nevi. According to a preferred embodiment of the present invention MM is differentiated from and other nevi according to the characteristic distribution of the melanocytes. In MM, the melanocytes are known to be arranged in “nests” which are conglomerates of cells that tend to crowd next to the tips of the rete ridges [Braun-Falco et al., 2003, “Histopathological characteristics of small diameter melanocytic naevi,” J. Clin. Pathol. 56: 459-464].

The differentiation between the distribution melanocyte in MM and the distribution melanocyte in other nevi, can be done, for example, by applying a set-operator which calculates one or more correlation functions. Melanocytes in MM are preferably identified when the distance auto-correlation function ξ_{αα}(r) and/or a position-angle correlation function, Ψ, has a local peak value next to the characteristic size of the nests. Alternatively or additionally, the melanocytes in MM are identified when the respective correlation function is relatively weaker than the correlation function of well-ordered tissues.

Since there is a mathematical connection between the n^{th} point correlation function and the distributions P_{R}(N) or P_{N}(R), the melanocytes in MM can also be identified based on these distributions. This is particularly useful when the correlation function does not fully describe the distribution. If, for example the probability distribution P(n) significantly differs from a Gaussian distribution, the use of P_{R}(N) or P_{N}(R) for MM identification is preferred over the use of correlation functions.

A common feature of melanocytes within the dermis is their tendency to “mature” while they migrate to deeper layers of the dermis towards the subcutaneous fat. The manifestation of this process in a benign skin lesion is the decrease in the cell nucleous size as function of its distance from the dermal-epidermal junction. This quantity can be displayed in accordance with preferred embodiments of the present invention as the average (modeled or non-modeled) cell nucleus size as function of its distance from either the junction or the straits corneum. FIG. 8b, for example, shows an unexpected behavior where the average nucleus size is slightly increased with the invasion depth. If such a trend is statistically significant and reoccurs in many ρ-columns of the nevus it may indicate malignancy.

Prominent nucleoli within the melanocyte nuclei are common feature of MM and are rather rare in benign cells. The present embodiments provide more than one way to reveal nucleoli existence.

In one preferred embodiment, sub-clustering is performed on the basis of the individual pixel normalization factor, Rᵢⱼ. Subsequently a set-operator which calculates a multiplicity function, which can be defined as the number of sub-clusters within each identified nucleus (e.g., cluster), is applied. The set-operator can also calculate the mean and variance of the multiplicity function. According to the presently preferred embodiment of the invention the number of nucleoli is proportional to the multiplicity function.

In another preferred embodiment, a set-operator which calculates the normalization factor variance within each identified nucleus is applied. According to the presently preferred embodiment of the invention the number of nucleoli is proportional to the normalization factor variance. This embodiment is particularly useful when the magnification level does not allow the detailed spatial analysis sub-clusters with similar normalization factors.

The present embodiments also contemplate the characterization of MM progression by means of rete ridges. In accordance with preferred embodiments of the present invention the existence and extent of rete ridges can be determined by applying a set-operator which calculates fractal dimension of the dermal-epidermal junction. The fractal dimension can be expressed as the ratio of line length per unit θ length, where θ is the complementary coordinate to ρ. The mean curvature of that line can also be used as a measure to the complexity of the line. According to a preferred embodiment of the present invention the more fractal-like is the dimension of the dermal-epidermal junction, or the higher the ratio of the line length with respect to the average epidermis width, the more malignant is the nevus.

Another expression for a general disorder of the tissue is the lack of directional correlation. In various exemplary embodiments of the invention the MM is identified by applying a set-operator which calculates a directional quantity, such as, but not limited to, the correlation functions ξ_{αβ}(ρ) and ξ_{αβ}(θ). The level of correlation is inversely proportional to the malignancy of the melanocyte. When only short range correlation or no correlation is found, the tissue is identified as MM.

There are several (micro) stages in the progress of MM. In the early stages, the melanocytes are located mostly at the dermal epidermal junction and appear almost equidistant from one another. In the intermediate stages, melanocytes proliferation occurs, at the beginning near the dermal-epidermal junction, and later in the upper layers of the epidermis and atypical nuclei (both size and shape) are formed. These stages are accompanied by formation of nests. In the advanced stages, the nests become larger, appear in the epidermis as well and the melanocytes occupy the dermis and become confluent.

The present embodiments successfully provide mathematical signatures which can be used for the identification of each of the various micro-stages. Broadly speaking, the MM progress can be characterized by several functions. When the melanocyte local density is employed, the early stages of MM progress are identified when the melanocyte local density has a peak which is narrower than a predetermined threshold, and the later stages are identified when melanocyte local density has a wider peak or has a generally flat shape as a function of r.

When an angular correlation function is employed, the early stages of MM progress are identified when the angular correlation function is above a predetermined threshold, and the later stages are identified the angular correlation function is below the predetermined threshold.

When the size distribution function P(n) is employed, the early stages of MM progress are identified when the width of P(n) is narrower than a predetermined threshold and the later stages are identified when the width of P(n) is wider than the predetermined threshold.

When both size distribution function P(n) and the GoF distribution for a specific geometrical model is employed, the early stages of MM progress are identified when the locally measured correlation between P(n) and GoF distribution width is higher than a predetermined threshold. Namely, more melanocyte-crowded regions are populated by relatively deformed nuclei.

When the P_{R}(N) distribution is employed with D being on the scale of a few melanocyte diameters, intermediate stages of the MM progress are identified when P_{R}(N), has two peaks at N≈1 and N≈3-5, and advanced stages are identified when P_{R}(N) is substantially flat or has one weak peak at the value N≈1.

Following is a more detailed description of the various stages in the MM progress, together with the preferred mathematical signatures of each stage.

Hence, in a benign nevus, the melanocytes are located mostly at the dermal epidermal junction and appear almost equidistant from one another. This stage can be identified by calculating the melanocyte local density along the internal ρ coordinate as defined earlier. The first stage is identified according to preferred embodiments of the present invention when the melanocyte local density (P(n), for different strips of ρ) has a peak which is correlated with the local epidermis thickness calculated as function of the same coordinate. The first stage can also be identified when there is a correlation of melanocytes as function of their angular coordinate θ in a coordinate system defined, e.g., with respect to the tissue boundary or the dermis-epidermis junction line coordinate. Equivalently, the first stage can be identified a prominent peak at approximately 1/θ₀ in the Fourier transform of the angular correlation function, where θ₀ is the typical inter-melanocyte distance. Alternatively the correlation can be calculated on the one-dimensional dermal-epidermal junction line in which case the correlation peak is obtained at approximately (δθ₀)⁻¹, with δ being the typical epidermal distance from the coordinate origin.

In a second stage, melanocytes are arranged non uniformly at the same location. The identification of this stage, according to preferred embodiments of the present invention is similar to the identification of the first stage, but with a weaker angular (or line) correlation function (wider Fourier transform).

In the third stage of MM, melanocytes proliferation begins. The proliferation is mostly confined to the dermal epidermal junction but some outliers start migrating upwards to the epidermis. A few atypical nuclei (both size and shape) are also formed. A signature for the third stage can be a widening of the peak in the melanocytic local density function (as function of ρ). The melanocytic local density in this stage increases inside the epidermis, namely at ρ values that are smaller than the local epidermis width. In addition, the two-point spatial correlation function is weaker and wider. Another characterization of the third stage is the appearance of outliers in the distribution function P(n) of the melanocytes, and in the GoF distribution of the geometric model, where there is a correlation between the populations of the two types of outliers. The third stage of the MM development can therefore be identified, in accordance with preferred embodiments of the present invention by calculating the distributions P(n) and GoF, identifying and outliers therein and calculating the correlation between the outliers, where high correlation correspond to third MM stage.

A common signature for the first, second and third stages can be negative (anti) correlation at scales of single melanocytes indicating the rareness of overlapping cells.

In a fourth stage, the melanocytes continue to migrate to the upper layers of the epidermis and nests are formed at the dermal-epidermal junction. A signature for this stage can be a further widening of the peak in the melanocytic local density function as a function of ρ. Another signature for this stage can be positive correlation function at scales of single melanocytes. An additional signature for the fourth stage is the appearance of two peaks in the P_{R}(N) distribution, for R values which correspond to a few melanocyte scales. A first peak is typically at the value of N≈1 and a second peak is typically obtained at the values N≈3-5. An additional signature for this stage can be the appearance of more outliers in the P(n) and GoF distributions, compared to the third stage. A further signature of the fourth step can be a change in the eccentricity distribution, compared to the first, second and third stages.

The fifth stage of MM development is an advanced expression of the fourth stage. In this stage, nests also appear at the epidermis, the nests are larger and the melanocytes are confluent. The signatures of the fifth stage are generally similar to the signatures of the fourth stage, but the appearance is more prominent. In addition, the wider distribution function P(n) for different ρ strips in the fifth stage is no longer restricted to a strip about the junction. Another signature of the fifth stage is the appearance of a flatter P_{R}(N) distribution. Specifically, whereas in the forth stage P_{R}(N) is characterized by two peaks, in the fifth stage P_{R}(N) is substantially flat with a possible peak at the value “N≈1”.

In a sixth stage, the melanocytes descend into the dermis and the nests become larger. There appears to be is no spatial arrangement of the melanocytes apart from higher density (of single melanocytes and nests of melanocytes) near to the dermal-epidermal junction. Signatures for the sixth stage include (i) flatter melanocyte local density function, (ii) wider behavior of P(n) with more prominent tails, compared to the other stages, (iii) P_{R}(N) shows log-normal or similar behavior with increasing R (from a few to many melanocytes scales), and (iv) large scatter in eccentricity and model GoF values.

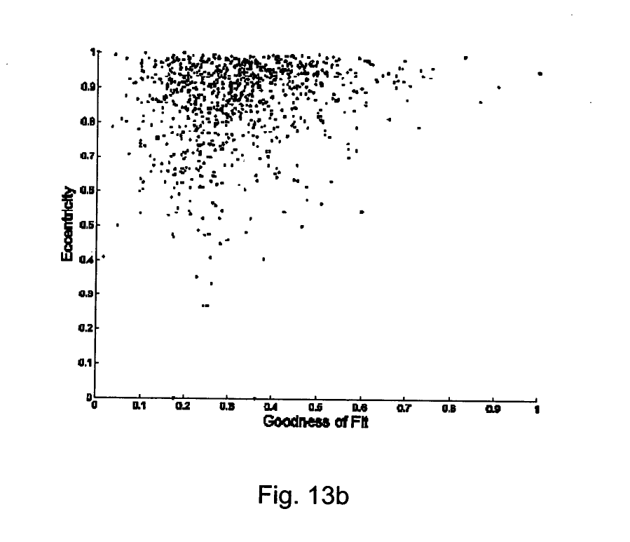

FIGS. 13a-b show two examples of the bi-variant distribution of skin lesion nuclei eccentricity and GoF of the geometrical model. The correlation between the eccentricity and the GoF is evident (see also FIG. 5b). It is therefore demonstrated that the present embodiments are suitable for characterizing skin lesion sections.

Beyond the aforementioned stages MM can progress to deeper skin layers, such as the subcutaneous layer and deeper. The present embodiments successfully track the invasion depth by means of similar mathematical signatures as described above.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A method of analyzing an image of a stained pathological specimen, the image being arranged gridwise in a plurality of picture-elements, each being associated with image data over a grid, the method comprising: defining at least one set of picture-elements over said grid, and applying, on each set of picture-elements, at least one set-operator, wherein each set-operator is associated with a predetermined diagnosis describing the pathological specimen, thereby analyzing the image.

2. The method of claim 1, further comprising issuing a report describing the pathological specimen, based on results obtained by said application of said at least one set-operator.

3. The method of claim 1, wherein said at least one set-operator comprises an operator for calculating statistical distributions.

4. The method of claim 1, wherein said at least one set-operator comprises an operator for calculating statistical moments.

5. The method of claim 1, wherein said at least one set-operator comprises an operator for calculating tensor of inertia.

6. The method of claim 1, wherein said at least one set-operator comprises an operator for calculating coordinates.

7. The method of claim 1, wherein said at least one set-operator comprises an operator for calculating population characteristics of said at least one set of picture-elements, hence to provide a population map characterizing the stained pathological specimen.

8. A method of characterizing a stained pathological specimen, the method comprising:
obtaining an image of the specimen, said image being arranged gridwise in a plurality of picture-elements each being associated with image data;
classifying said picture-elements into classification groups according to said image data;
using said classification groups to define at least one set of picture-elements corresponding to at least one tissue region of the pathological specimen; and
applying, on each set of picture-elements, at least one set-operator so as to characterize said tissue regions according to image data and spatial characteristics of said set;
thereby characterizing the pathological specimen.

9. The method of claim 8, further comprising using said classification groups to define at least one set of picture-elements corresponding to at least one background region of the pathological specimen.

10. The method of claim 8, wherein said defining said at least one set of picture-elements comprises clustering at least a portion of said picture-elements according to said classification groups, thereby providing at least one cluster of picture-elements.

11. The method of claim 10, wherein said applying said at least one set-operator on each said set of picture-elements, comprises applying said at least one set-operator on said at least one cluster of picture-elements.

12. The method of claim 8, wherein said defining said at least one set of picture-elements comprises applying a geometrical modeling procedure to at least a portion of said plurality of picture-elements.

13. The method of claim 10, wherein said defining said at least one set of picture-elements comprises applying a geometrical modeling procedure to said at least one cluster of picture-elements.

14. The method of claim 8, further comprising normalizing said image data prior to said classification.

15. The method of claim 8, further comprising combining at least a portion of said classification groups.

16. The method of claim 8, further comprising employing at least one counting technique to the stained pathological specimen, and correlating the results of said counting technique with said at least one set of picture-elements.

17. Apparatus for characterizing a stained pathological specimen based on an image of the specimen, the image being arranged in a plurality of picture-elements each being associated with image data, the apparatus comprising:
classification unit, for classifying said picture-elements into classification groups according to said image data;
a set definition unit, for defining at least one set of picture-elements corresponding to at least one tissue region of the pathological specimen, using said classification groups;
a data analysis unit, for applying at least one set-operator on each set of picture-elements, so as to characterize said tissue regions according to image data and spatial characteristics of said set, thereby characterizing the pathological specimen.

18. A system for characterizing a stained pathological specimen, comprising an imaging apparatus, for providing the image of the specimen, and the apparatus of claim 17.

19. The apparatus of claim 17, wherein said set definition unit is operable to define at least one set of picture-elements corresponding to at least one background region of the pathological specimen, using said classification groups.

20. The apparatus of claim 17, further comprising a clustering unit, for clustering at least a portion of said picture-elements according to said classification groups, to provide at least one cluster of picture-elements.

21. The apparatus of claim 20, wherein said data analysis unit is operable to apply said at least one set-operator on said at least one cluster of picture-elements.

22. The apparatus of claim 17, further comprising a geometrical modeling unit for applying a geometrical modeling procedure to at least a portion of said plurality of picture-elements.

23. The apparatus of claim 20, further comprising a geometrical modeling unit for applying a geometrical modeling procedure to said at least one cluster of picture-elements.

24. The apparatus of claim 17, wherein said classification unit is operable to normalize said image data.

25. The apparatus of claim 17, wherein said classification unit is operable to combine at least a portion of said classification groups.

26. The apparatus of claim 20, wherein said at least one cluster comprises at least one sub-cluster.

27. The apparatus of claim 26, wherein said geometrical modeling is applied to said at least one sub-cluster.

28. The apparatus of claim 20, wherein said at least one cluster comprises at least one cluster of background picture-elements.

29. The apparatus of claim 28, wherein said at least one set of picture-elements is defined by cross correlation of at least one cluster of background picture-elements with at least one cluster of non-background picture-elements.

30. The apparatus of claim 17, wherein said image comprises a spectral image and said image data comprises a wavelength spectrum.

31. The apparatus of claim 17, wherein said image comprises a monochrome image and said image data comprises intensity values.

32. The apparatus of claim 30, wherein said classification groups are selected from a predefined set of classification groups, each being associated with a predetermined wavelength spectrum.

33. The apparatus of claim 30, wherein said classification groups are defined based on the wavelength spectra of said picture-elements.

34. The apparatus of claim 33, wherein said classification groups are defined iteratively.

35. The apparatus of claim 33, wherein said classification groups are defined non-iteratively.

36. The apparatus of claim 17, wherein said at least one set-operator comprises an operator for calculating statistical distributions.

37. The apparatus of claim 17, wherein said at least one set-operator comprises an operator for calculating statistical moments.

38. The apparatus of claim 17, wherein said at least one set-operator comprises an operator for calculating tensor of inertia.

39. The apparatus of claim 22, wherein said at least one set-operator comprises an operator for calculating distribution of parameters obtained from said geometrical modeling procedure.

40. The apparatus of claim 17, wherein said at least one set-operator comprises an operator for calculating coordinates.

41. The apparatus of claim 20, wherein said at least one set-operator comprises an operator for calculating an average normalization factor over said at least one cluster of picture-element.

42. The apparatus of claim 17, wherein said at least one set-operator comprises an operator for calculating population characteristics of said at least one set of picture-elements, hence to provide a population map characterizing the stained pathological specimen.

43. The method of claim 42, further comprising employing at least one counting technique to the stained pathological specimen, to provide an amplification map characterizing the stained pathological specimen, and correlating said amplification map with said population map.

44. The apparatus of claim 30, wherein said spectral image is characterized by two spatial dimensions.

45. The apparatus of claim 30, wherein said spectral image is characterized by three spatial dimensions.

46. The apparatus of claim 17, wherein said image comprises a set of spectral images and said image data comprises a wavelength spectrum.

47. The apparatus of claim 46, wherein at least two images of said set of spectral images are characterized by a different magnification level.

48. The apparatus of claim 46, wherein at least two images of said set of spectral images are captured following a different staining of the pathological specimen.

49. The apparatus of claim 46, wherein at least two images of said set of spectral images are captured by a different illumination scheme.

50. The apparatus of claim 46, wherein at least two images of said set of spectral images are captured by a different spectral acquisition scheme.

51. The apparatus of claim 46, wherein at least two images of said set of spectral images correspond to different region-of-interests of the pathological specimen.

52. The apparatus of claim 17, wherein the pathological image is stained with a stain selected from the group consisting of a direct immunohistochemical stain, a secondary immunohistochemical stain, a histological stain, immunofluorescence stain, a DNA ploidy stain, a nucleic acid sequence specific probe and any combination thereof.

53. The apparatus of claim 17, wherein the pathological image is stained using a method selected from the group consisting of Romanowsky-Giemsa staining, Haematoxylin-Eosin staining and May-Grunwald-Giemsa staining.
